(19) United States
(12) Patent Application Publication
(10) Pub. No.: US 2024/0024675 A1
(43) Pub. Date: Jan. 25, 2024

(54) VESTIBULAR STIMULATION MEDICAL DEVICE

(71) Applicant: Medtronic Xomed, LLC, Minneapolis, MN (US)

(72) Inventors: Alex Santo Tarantino, Plymouth, MN (US); Mirko de Melis, Maastricht (NL)

(21) Appl. No.: 18/351,353

(22) Filed: Jul. 12, 2023

Related U.S. Application Data
(60) Provisional application No. 63/369,112, filed on Jul. 22, 2022.

(51) Int. Cl. A61N 1/36

(52) U.S. Cl. CPC A61N 1/36036; A61N 1/36139

(57) ABSTRACT

A vestibular implant system including a memory; an implantable sensor; a vestibular implant; and processing circuitry coupled to the memory. The processing circuitry is configured to: detect, via the implantable sensor, an electrical signal of a patient that is generated in response to an electrical stimulation signal transmitted by the vestibular implant to one or more regions of vestibular organ of a patient; determine, based on the detected electrical signal, that the vestibular implant is transmitting the electrical stimulation signal at a first rate of change of an amplitude of the electrical stimulation signal; and based on a determination that the first rate of change satisfies a threshold condition, adjust one or more parameters of the electrical stimulation signal to cause the vestibular implant to transmit the electrical stimulation signal to the nerve at a second rate of change of the amplitude of the electrical stimulation signal.

This application claims the benefit of U.S. Provisional Patent Application No. 63/369,112, filed Jul. 22, 2022 and entitled “VESTIBULAR STIMULATION MEDICAL DEVICE,” the entire contents of which is incorporated herein by reference.

### TECHNICAL FIELD

The disclosure relates to medical device systems, and more particularly, to medical device systems for delivery of electrical stimulation therapy.

### BACKGROUND

A vestibular system of a patient may detect movements and/or accelerations of the head of the patient and provide sensory information to assist the body of the patient in maintenance of balance, orientation, and motor output. The vestibular organ is located within the temporal bone and includes the inner ear including the cochlea, the vestibule and semicircular canals (anterior, posterior, and lateral), and the membranous labyrinth. The vestibular organ translates head movement into neural output and transmits the neural output to the central nervous system of the patient. The central nervous system may produce motor output instructions to the body of the patient and one or more reflexes to adjust body positioning of the patient

Injuries to a patient's vestibular system may impair the functions of the vestibule, semicircular canals, and/or the membranous labyrinth and may affect the patient's sense of balance. Damage to the vestibular system may lead to dizziness, decreased balance, changes in vision and/or hearing, attacks of vertigo, or the like. Electrical stimulation to the vestibular system (e.g., semicircular canals, vestibular nerves, otolith organs, membranous labyrinth) by a medical device may improve the patient's symptoms.

### SUMMARY

In general, the devices, systems, and techniques of this disclosure generally relate to an implantable medical device (IMD) system and methods for therapy for disorders and/or impairment of a vestibular system of a patient but can be extended to address other patient symptoms and disorders. Some example techniques include electrical stimulation of one or more semicircular canals, otolith organs, vestibular nerves, and/or regions of the membranous labyrinth of the vestibular organ of a patient to improve the patient's sense of balance and/or reduce the effects of symptoms of the disorder and/or impairment. Some example techniques include closed-loop electrical stimulation of regions of the vestibular organ

To stimulate the vestibular organ, a medical device (e.g., an IMD) outputs electrical simulation signals via one or more electrodes on one or more implanted leads to regions of the vestibular organ to improve the patient's sense of balance. The stimulated regions of the vestibular organ may correspond to particular ranges of motion of the patient's head (e.g., horizontal movement, vertical movement, or the like). For example, stimulation to a first region of the vestibular organ may cause the vestibular organ to generate a signal indicating head movement in a first direction, stimulation to a second region of the vestibular organ may cause the vestibular organ to generate a signal indicating head movement in a second direction, and so forth. In some examples, such as with an IMD, a clinician may form a channel (e.g., within the temporal bone) from the IMD implanted within the patient (e.g., within a subcutaneous pocket) to the vestibular organ and connect the vestibular organ to the IMD via one or more electrical leads disposed within the channel.

In some examples, this disclosure describes example implantation devices, systems, and techniques for connecting the electrical leads to regions of the vestibular organ of the patient (e.g., one or more semicircular canals, otolith organs, or the like). In some examples, this disclosure describes example devices, systems, and techniques for adjusting the transmitted electrical stimulation signals to the vestibular organ based on sensor information from one or more sensors of the medical device system corresponding to movement of the patient. In some examples, this disclosure describes example devices, systems, and techniques for adjusting the transmitted electrical stimulation signals to the vestibular organ based on sensor information from one or more sensors of the medical device system corresponding to the effects of the transmitted electrical stimulation signal on the patient. The various example techniques described in this disclosure may be performed in combination or may be performed separately.

In one example, this disclosure is directed to a vestibular implant system comprising: a memory; an implantable sensor; a vestibular implant; and processing circuitry coupled to the memory, the processing circuitry configured to: detect, via the implantable sensor, an electrical signal of a patient that is generated in response to an electrical stimulation signal transmitted by the vestibular implant to one or more regions of vestibular organ of a patient; determine, based on the detected electrical signal, that the vestibular implant is transmitting the electrical stimulation signal at a first rate of change of an amplitude of the electrical stimulation signal; and based on a determination that the first rate of change satisfies a threshold condition, adjust one or more parameters of the electrical stimulation signal to cause the vestibular implant to transmit the electrical stimulation signal to the nerve at a second rate of change of the amplitude of the electrical stimulation signal.

In another example, this disclosure is directed to a method comprising: transmitting, via a vestibular implant of a vestibular implant system, an electrical stimulation signal to one or more regions of vestibular organ of a patient; detecting, via an implantable sensor of the vestibular implant system, an electrical signal of a patient in response to the transmitted electrical stimulation signal; determining, by processing circuitry of the vestibular implant system and based on the detected electrical signal, that the vestibular implant is transmitting the electrical stimulation signal to a nerve at a first rate of change of amplitude of the electrical stimulation signal; determining, by the processing circuitry, whether the first rate of change of the amplitude satisfies a threshold condition; and adjusting, based on the determination that the first rate of change of the amplitude satisfies the threshold condition, one or more parameters of the electrical stimulation signal to cause the vestibular implant to transmit the electrical stimulation signal to the nerve at a second rate of change of the amplitude.

In another example, this disclosure is directed to a vestibular implant system comprising: a vestibular implant; and a plurality of electrical leads, each of the plurality of electrical leads comprising: an elongated body extending from a proximal end to a distal end; an electrical conductor disposed on the proximal end of the elongated body and connected to the vestibular implant; an electrode disposed on the distal end of the elongated body; and an attachment device disposed on the distal end of the elongated body, wherein the attachment device is configured to affix the electrode to a semicircular canal of a patient at an implantation site on the semicircular canal.

In another example, this disclosure is directed to a method comprising: navigating a drill to temporal bone of a patient; forming a channel in the temporal bone to a recess surrounding a semicircular canal of the patient; advancing a catheter of a vestibular implant system through the channel and into the recess; disposing, via the catheter, an electrical lead within the recess, wherein the electrical lead is connected to a vestibular implant; and affixing the electrical lead to the base of the semicircular canal.

This summary is intended to provide an overview of the subject matter described in this disclosure. It is not intended to provide an exclusive or exhaustive explanation of the methods and systems described in detail within the accompanying drawings and description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual diagram illustrating an implantable medical device (IMD) system for delivering electrical stimulation signals to a vestibular organ.

FIG. 2 is a block diagram illustrating an example configuration of an example implantable medical device (IMD) of the system of FIG. 1.

FIG. 3 is a conceptual diagram illustrating connections of electrical leads of the system of FIG. 1 to the vestibular organ.

FIG. 4A is a conceptual diagram illustrating an example location for the IMDs of the system of FIG. 1.

FIG. 4B is a conceptual diagram illustrating another example location of the IMDs for the system of FIG. 1.

FIG. 5 is a conceptual diagram illustrating example electrical leads of FIG. 1 disposed within a channel in a temporal bone of the patient.

FIGS. 6A-F illustrate example attachment devices of the system of FIG. 1.

FIG. 7 is a conceptual diagram illustrating example stimulation modes of the system of FIG. 1.

FIG. 8 is a conceptual diagram illustrating other example stimulation modes of the system of FIG. 1.

FIG. 9 is a conceptual diagram illustrating an example user interface (UI) displaying an example process of adjusting electrical stimulation signals transmitted by the system of FIG. 1.

FIG. 10 is a flow diagram illustrating an example process of delivering electrical stimulation signals to the vestibular organ of the patient.

FIG. 11 is a flow diagram illustrating an example process of adjusting electrical stimulation signals based on patient response.

FIG. 12 is a flow diagram illustrating an example process of implanting the system of FIG. 1.

### DETAILED DESCRIPTION

Medical devices, systems, and techniques for delivering electrical stimulation signals (also referred to as “electrical stimulation signals”) to regions of a vestibular organ of a patient are described in this disclosure. Electrical stimulation signals are delivered to improve balance of the patient. Electrical stimulation signals may also be delivered to reduce the effects of symptoms of injury and/or impairment to the vestibular organ. The symptoms may include, but are not limited to, dizziness, decreased balance, changes in vision and/or hearing, or attacks of vertigo.

This disclosure describes example implantation devices, systems, and techniques for connecting the electrical leads to regions of the vestibular organ of the patient (e.g., one or more semicircular canals, otolith organs, or the like). In some examples, this disclosure describes example devices, systems, and techniques for adjusting the transmitted electrical stimulation signals to the vestibular organ based on sensor information from one or more sensors of the medical device system corresponding to movement of the patient. In some examples, this disclosure describes example devices, systems, and techniques for adjusting the transmitted electrical stimulation signals to the vestibular organ based on sensor information from one or more sensors of the medical device system corresponding to the effects of the transmitted electrical stimulation signal on the patient.

The example implantation devices, systems, and techniques provide one or more improvements over other example medical devices configured to transmit electrical stimulation signals to the vestibular organ. Adjustment of the transmitted electrical stimulation signals based on patient movement may reduce unnecessary stimulation when the patient is performing activities and/or is in a position that does not require a higher level of stimulation and may extend the lifespan of a power source of the example medical device system. Additionally, the adjustment of the transmitted electrical stimulation signals based on patient movement may improve efficacy of the electrical stimulation signals in improving patient balance and/or reducing symptom effects. For example, the adjustment of the transmitted electrical stimulation may allow for dynamic adjustment of the electrical stimulation signal to match patient activity when the patient is transitioning between multiple activity states (e.g., from resting to exercising within a short period of time). Adjustment of the transmitted electrical stimulation signals based on patient response to the electrical stimulation signals may improve efficacy of therapy by reducing an amount of unintended stimulation experienced by the patient and reducing a time period between initiation of the electrical stimulation signal and when the patient may experience the effects of the electrical stimulation signal.

The implantation techniques described in this disclosure may simplify the implantation process by providing a common access point through a channel (e.g., at the base of the semicircular canals) to the vestibular organ instead of requiring individual access points for each electrical lead of a medical device system. The attachment devices described in this disclosure may facilitate easier affixation and removal of electrical leads from the vestibular organ of the patient.

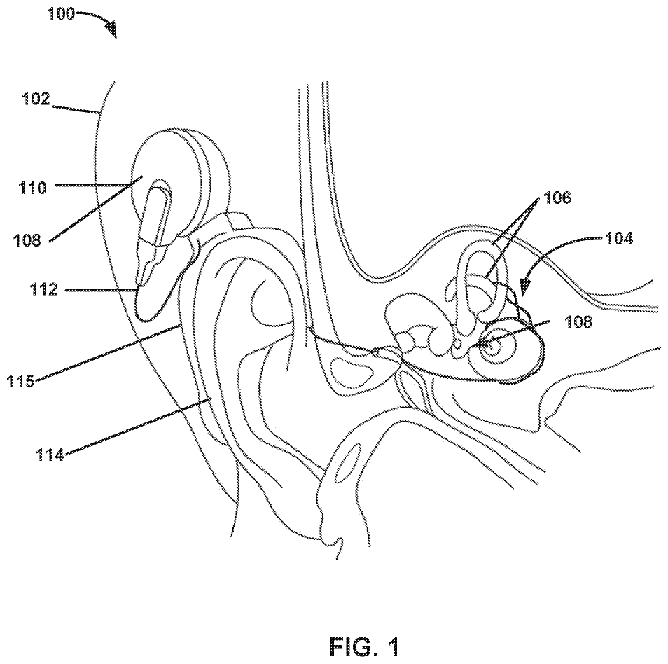

FIG. 1 is a conceptual diagram illustrating an implantable medical device (IMD) system 100 for delivering electrical stimulation signals to a vestibular organ 104. Medical device system 100 may include IMD 108 and electrical lead(s) 112. In some examples IMD system 100 may be a vestibular implant system.

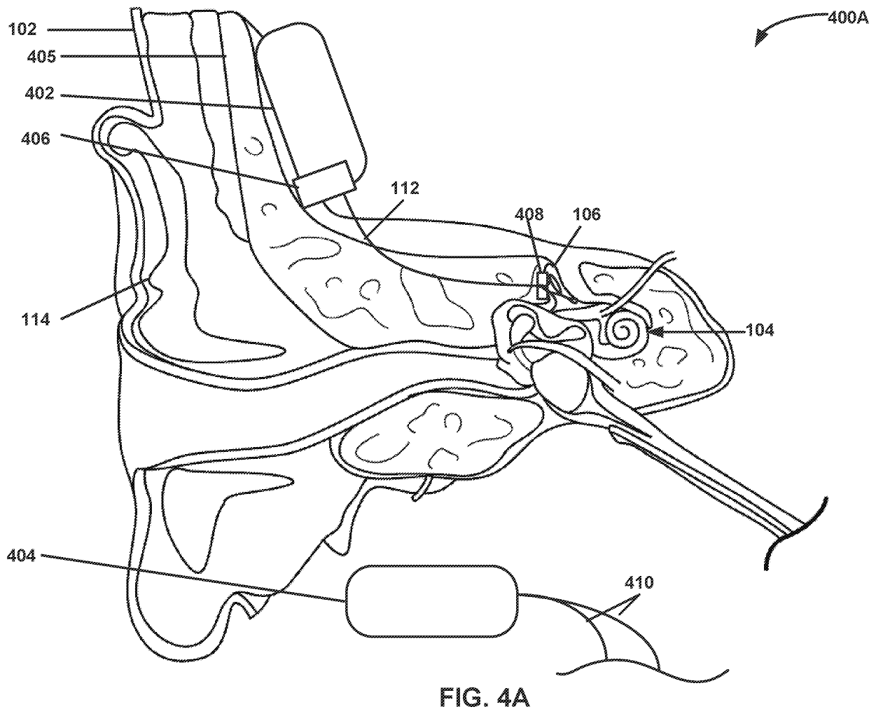

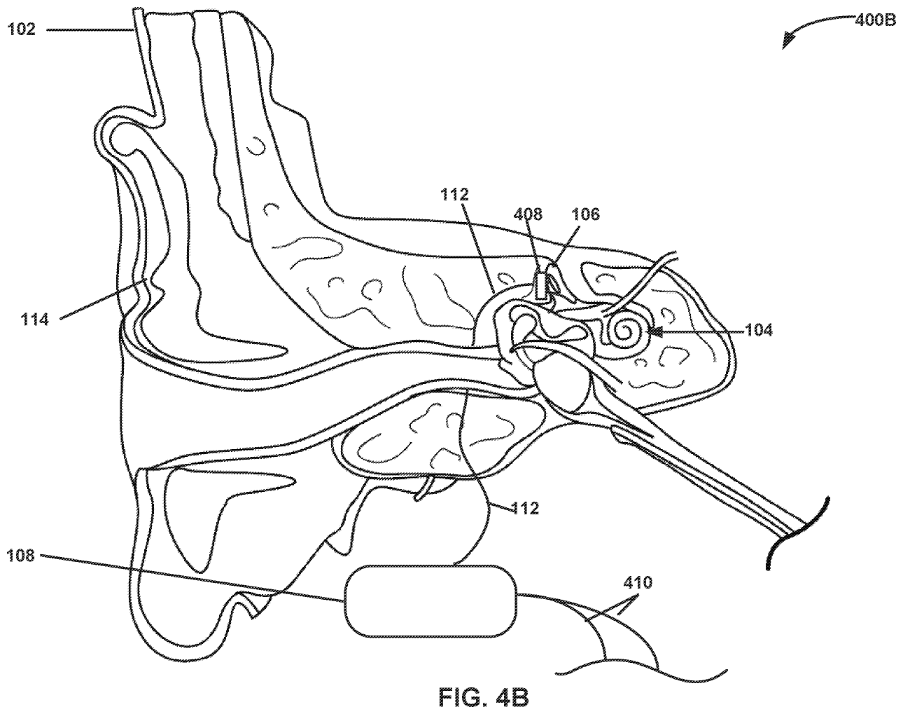

IMD 108 may be implanted within the head of patient 102. Electrical lead(s) 112 is disposed within the patient and is connected to regions of vestibular organ 104. In some examples, as illustrated in FIG. 1, IMD 108 may be implanted behind ear 114 of patient 102 and may be connected to ear 114 via an earpiece 115 disposed around ear 114. Earpiece 115 may be integral to or separate from housing 110 of IMD 108. In other examples, as discussed in greater detail with respect to FIGS. 4A and 4B below, IMD 108 may be one or more devices implanted within patient 102 at other locations (e.g., within the cranial cavity, within the neck, or like).

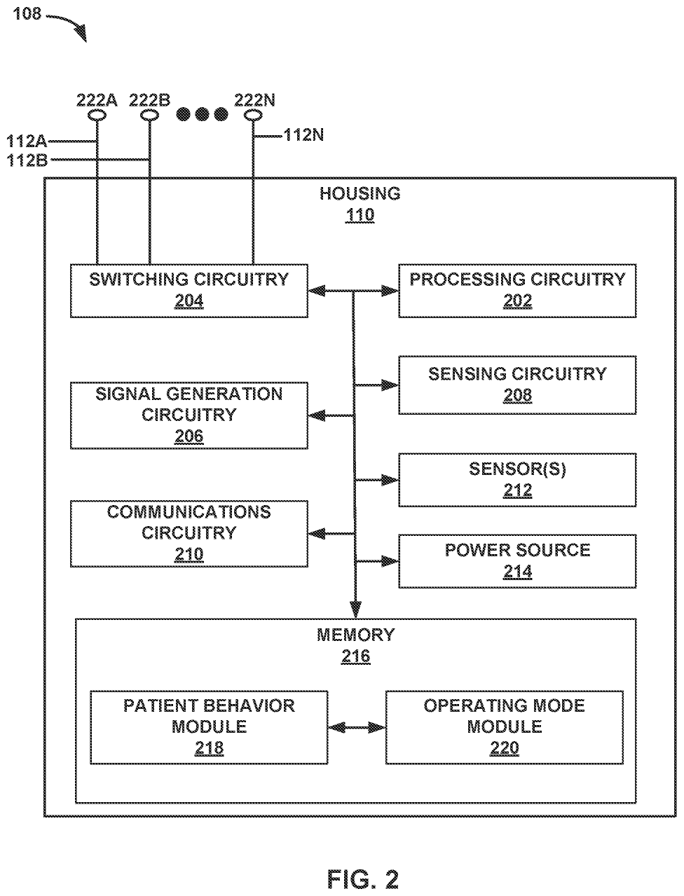

As discussed in greater detail in FIG. 2, IMD 108 includes a plurality of sensors and computer circuitry configured to perform the example processes and techniques described within this disclosure. IMD 108 may generate, e.g., via the computer circuitry of IMD 108, an electrical stimulation signal and transmit the electrical stimulation signal to vestibular organ 104. The sensors and computer circuitry are disposed within housing 110 implanted into patient 102. Sensors and/or computer circuitry may be connected to electrical lead(s) 112 through one or more ports within housing 110.

Vestibular organ 104 is disposed within the temporal bone of patient 102 and includes vestibule 108, semicircular canals 106. Vestibular organ 104 is a part of an inner ear of patient 102 and is disposed behind cochlea of patient 102. Vestibule 108 may contain a region of the membranous labyrinth and otolith organs (sacculus and utricle). Semicircular canals 106 are connected to vestibule 108 and include horizontal semicircular canal, superior semicircular canal, and posterior semicircular canal. Vestibule 108 and semicircular canals detect movement and/or rotation of the head of patient 102, translates the detected movement and/or rotation into neural output, and transmits the neural output to central nervous system (e.g., the brain) of patient 102 through vestibular nerves connected to vestibule 108 and semicircular canals 106. Damage and/or impairment to vestibule 108 and/or semicircular canals 106 may lead to an inability of vestibular organ 104 to accurately detect movement and/or rotation of the head of patient 102, translate the detected movement and/or rotation into the appropriate neural outputs, and/or transmit the neural outputs to the central nervous system of patient 102.

IMD system 100 may detect movement of the head of patient 102 via one or more sensors (e.g., accelerometer, gyroscope, implantable sensors within muscle of patient 102, or the like). Based on the detected movement of the head, IMD system 100 may transmit electrical stimulation signals via electrical lead(s) 112 to the corresponding regions of vestibular organ 104 that detect the types of movement (e.g., movement about a particular plane, rotation about a particular plane). Electrical lead(s) 112 may transmit the electrical stimulation signals to the corresponding regions of vestibular organ 104 and into the connected vestibular nerves. In some examples, the electrical stimulation signals may cause the stimulated regions of vestibular organ 104 to generate a corresponding neural output and transmit the neural output to the vestibular nerve. In some examples, the electrical stimulation signals may be the neural output that a functioning vestibular organ outputs in response to the detected movement. As such, IMD system 100 may form a close-loop system to deliver stimulation to patient 102.

In some examples, patient 102 does not require constant stimulation from IMD system 100. IMD system 100 may control whether and when to deliver electrical stimulation signals to patient 102, e.g., based on motion data and/or sensor data detected by IMD system 100. In some examples, IMD system 100 may determine whether and when to deliver electrical stimulation signals via a closed-loop system.

In some examples, IMD system 100 may store a plurality of operating modes. Each operating mode may define the parameters of the electrical stimulation signal including example parameters such as the amplitude of the electrical stimulation signal, the frequency of the electrical stimulation signal, and/or which regions of vestibular organ 104 to (e.g., one or more semicircular canals 106, one or more otolith organs of vestibule 108) to prioritize or avoid transmission of the electrical stimulation signal. In some examples, IMD system 100 operating under an operating mode made transmit electrical stimulation signals with different parameters to different regions of vestibular organ 104. In some examples, each operating mode defines a range of possible values for each parameter (e.g., a maximum amplitude, a minimum amplitude, a maximum amplitude to one of semicircular canals 106, or the like).

In some examples, IMD system 100 may include implantable sensors connected to muscles and/or nerves of patient 102. The implantable sensors may detect electrical signals corresponding to the response of patient 102 to a transmitted electrical stimulation signal. The electrical stimulation signals corresponding to the response of patient 102 may include electrical activity (e.g., evoked electrical stimulation signals) in a nerve of patient 102, and signal saturation metrics (e.g., signal saturation status, signal saturation degree, and/or signal saturation decay). IMD system 100 may use the detected electrical signals to adjust the parameters of the electrical stimulation signal, e.g., to increase efficacy of the electrical stimulation signal and/or reduce delivery of unintended electrical stimulation signals.

In some examples, as illustrated in FIG. 1, IMD 108 of IMD system 100 may be implanted behind ear 114 of patient 102 and externally on the head of patient 102. In other examples, as illustrated and described in greater detail in FIGS. 4A and 4B, IMD system 100 may include one or more IMD 108 implanted within tissue of patient 102 and/or within the cranial cavity of patient 102.

FIG. 2 is a block diagram illustrating an example configuration of example IMD 108 of IMD system 100 of FIG. 1. IMD 108 may include electrical leads 112A-N (collectively referred to as “electrical leads 112”), one or more of which may be configured as described with respect to FIG. 1. For example, electrical leads 112 may be configured to extend from housing 110 and be connected to regions of vestibular organ 104 (e.g., vestibule 108, one or more of semicircular canals 106). Each of electrical leads 112 may include a corresponding one of electrodes 222A-N (collectively referred to as “electrodes 222”) disposed on a distal end of the corresponding electrical lead 112. Electrodes 222 may be affixed to and/or placed in apposition to tissue of vestibular organ 104 and may deliver electrical stimulation signals from IMD 108 to the corresponding regions of vestibular organ 104.

In the example shown in FIG. 2, IMD 108 includes processing circuitry 202, switching circuitry 204, signal generation circuitry 206, sensing circuitry 208, communications circuitry 210 (also referred to as “telemetric circuitry 210”), sensor(s) 212, power source 214, and memory 216. The various circuitry may be, or include, programmable or fixed function circuitry configured to perform the functions attributed to respective circuitry. Memory 216 may store computer-readable instructions that, when executed by processing circuitry 202, cause IMD 108 to perform various functions. Memory 216 may be a storage device or other non-transitory medium. The components of IMD 108 illustrated in FIG. 2 may be housed within housing 110 or within a plurality of other housing. In some examples, a first IMD (e.g., a stimulator) is configured to deliver electrical stimulation signals in response to instructions from a second IMD. First IMD may include electrical leads 112, switching circuitry 204, signal generation circuitry 206, communications circuitry 210 configured to communicate with the second IMD, and power source 214. Second IMD may include processing circuitry 202 sensing circuitry 208, sensor(s) 212, communications circuitry 210, power source 214, and memory 216.

Signal generation circuitry 206 is configured to generate electrical stimulation signals at a range of amplitudes and at a range of frequencies. The range of frequencies may be between about 0 hertz (Hz) to about 1500 Hz. In some examples, the range of frequencies may be between about 200 Hz to about 1000 Hz. In some examples, electrical stimulation signal may include galvanic stimulation of vestibular organs 104. Galvanic stimulation signals may have an amplitude of between about 1.5 milliamps (mA) to about 5 mA. In some examples, the galvanic stimulation signals may include a sinusoidal profile having a frequency of between about 0.1 Hz to about 5 Hz. In some examples, the electrical stimulation signal may generate a sound output to vestibular organs 104, e.g., to improve balance of patient 102. The sound output may be between about 80 decibels (dB) to about 120 dB.

Signal generation circuitry 206 may include, as examples, current or voltage sources, capacitors, charge pumps, or other signal generation circuitry. Switch circuitry 204 is coupled to electrical leads 112 and may include one or more switch arrays, one or more multiplexers, one or more switches (e.g., a switch matrix or other collection of switches), one or more transistors, or other electrical circuitry. Switch circuitry 204 is configured to direct electrical stimulation signals from signal generation circuitry 206 to a selected combination of electrical leads 112, e.g., to selective deliver electrical stimulation signals to different regions of vestibular organ 104. For example, in response to IMD 108 operating in a first mode, switch circuitry 204 may couple electrical leads 112A and 112B to deliver electrical stimulation signals to horizontal semicircular canal of semicircular canals 106 and to the utricle of vestibule 108. One example of the first mode is where the patient is in a vehicle, and may be referred to IMD 108 operating in a “vehicle” mode to indicate the patient is in a vehicle.

However, in response to IMD 108 operating in a second mode, switch circuitry 204 may couple electrical leads 112A and 112B to deliver stimulation signals to a different region. Accordingly, in one or more example, IMD 108 may be configured to determine an operational mode based on sensed information, and deliver therapy accordingly.

Switch circuitry 204 may also selectively couple sensing circuitry 208 to electrical leads 112 and sensor(s) 212, e.g., to sense one or more electrical signals in tissue of patient 102 and/or to determine movement of the head of patient 102. Sensing circuitry 208 may include filters, amplifiers, analog-to-digital converters, or other circuitry configured to sense electrical signals in tissue of patient 102. In this manner, processing circuitry 202 may determine responses (e.g., evoked electrical signals) to electrical stimulation signals transmitted by electrical leads 112 to vestibular organ 104.

Sensor(s) 212 may include one or more accelerometers, gyroscopes, and one or more implantable sensors. Accelerometers may detect accelerations of the head of patient 102 and may transmit the acceleration data to sensing circuitry 208 and/or processing circuitry 202. Sensing circuitry 208 and/or processing circuitry 202 may determine, based on the acceleration data, an acceleration rate of the head of patient 102. Accelerometers may be one or more of a piezoelectric accelerometer, a piezoresistance accelerometer, or a capacitive accelerometer.

Gyroscopes may detect movement and/or rotation of the head of patient 102, e.g., by sensing an angular velocity of the head. Gyroscopes may include one or more vibration gyroscopes. The gyroscopes may detect rotation of head within one or more planes including the sagittal plane, the transverse plane, the coronal plane, or any combination herein. The gyroscopes may transmit the angular velocity data to sensing circuitry 208 and/or processing circuitry 202, which may then determine an angular shift and an angular shift frequency of the head of patient 102.

Implantable sensors may record electrical signals in tissue of patient 102. In some examples, implantable sensors are disposed near nerves and/or tissue within neck of patient 102. The implantable sensors may be disposed within and/or near the rectus muscles, the obliquus muscles, and the trapezius muscles. In some examples, the implantable sensors may be connected to one or more vestibular nerves and/or one or more nerves of the central nervous system of patient 102. The implantable sensors may detect evoked electrical signals (e.g., myogenic potential, cervical compound action potential (CCAP)), within the muscles in response to movement and/or rotation of the head of patient 102. In some examples, implantable sensors may be disposed near nerves in the head or neck region of patient 102 and may detect electrical signals (e.g., evoked or otherwise) corresponding to the transmitted electrical stimulation signals. The detected electrical signals may correspond to response of patient 102 to the transmitted electrical stimulation signals. The detected electrical signals may represent to a level of stimulation of a nerve, e.g., as reflected in electrical activity of the nerve or the myogenic potential transmitted by the nerve.

In some examples, the detected electrical signals may represent signal saturation metrics including, but are not limited to, signal saturation status, signal saturation degree, or signal saturation decay. Sensor(s) 212 may transmit the detected electrical signals to sensing circuitry 208 and/or processing circuitry 202. Signal saturation status may indicate whether the nerve is stimulated. Signal saturation degree may represent an amount of stimulation the nerve is receiving. In some examples, signal saturation degree may represent an amplitude of a detected electrical stimulation signal in the nerve relative to a range of possible electrical stimulation signal amplitudes. The range of possible electrical stimulation signal amplitudes may be based on maximum and minimum amplitudes for electrical stimulation signals for a particular operating mode IMD 108 is currently operating in. Signal saturation decay may represent a rate at which electrical charge leaves a nerve after termination of transmission of electrical stimulation signals to the nerve.

Processing circuitry 202 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 202 herein may be embodied as firmware, hardware, software, or any combination thereof.

Processing circuitry 202 may determine, based on motion data received from sensor(s) 212 (e.g., acceleration data, angular velocity data, detected electrical signals, or the like), movement of the head of patient 102 (e.g., a magnitude of the movement and frequency of the movement). Processing circuitry 202 may also determine a gait and/or posture of patient 102. In some examples, processing circuitry 202 may represent the motion data using one or more metrics including, but are not limited, an angular shift frequency, an acceleration rate, and an amplitude (e.g., a relative amplitude) of an evoked electrical signal (e.g., a myogenic potential). The evoked electrical signal may be a signal that is generated by tissue of patient 102 in response to delivery of an electrical signal (e.g., electrical stimulation signal). Sensor(s) 212 may detect evoked electrical signals in response to delivered electrical signals having voltages of between about 50 millivolts (mV) to about 10 volts (V). In some examples, the delivered electrical signal may have voltages of between about 100 mV to 5 V. In some examples, the evoked electrical signal may be generated in response to movement of patient 102. In some examples, the evoked electrical signal may exclude intrinsic electrical signals generated by the nerve of patient 102, but the techniques are not so limited in this disclosure. Processing circuitry 202 may retrieve reference values for each of the metrics from patient behavior module 218 of memory 216.

Processing circuitry 202 may compare the values of the one or more metrics, as determined based on the motion data, against the reference values for the one or more metrics to determine a current operating state of patient 102 (also referred to as “action” of patient 102). The operating state of patient 102 may correspond to actions performed by or on patient 102. Examples of the operating states patient 102 riding in a vehicle, exercising, sleeping, etc. The operating states may correspond to operating modes of IMD 108. For instance, in the vehicle operation state, IMD 108 may be considered to be in a vehicle operation mode. In the vehicle operation mode, IMD 108 may deliver stimulation to a first region with stimulation parameters within a first range. In the exercising state, IMD 108 may be considered to be an exercising mode. In the exercising mode, IMD 108 may deliver stimulation to a second, different region and/or with stimulation parameters within a second, different range.

For instance, processing circuitry 202 may then select, based on the determine operating state of patient 102, an operating mode from a plurality of operating modes stored in operating mode module 220. Each operating mode may include a range of possible stimulation parameters (e.g., range of amplitudes, range of frequencies), particular regions of ventricular organ 104 to favor, and/or particular regions of ventricular organ to disfavor and/or ignore. The selected operating mode may correspond to the activity level of the determined operating state of patient 102. For example, if processing circuitry 202 determines that patient 102 is currently in an operating state of exercise, processing circuitry 202 may select the operating mode of “dynamic exercise” from the plurality of operating modes stored in operating mode module 220.

Processing circuitry 202 may transmit instructions to switching circuitry 204, sensing circuitry 208, signal generation circuitry 206, and the like to deliver electrical stimulation signals with parameters within the range of parameters for the selected operating mode. For example, processing circuitry 202 may instruct switching circuitry 204 to deliver electrical stimulation signals with amplitudes and/or frequencies within the acceptable ranges for the operating mode and to prioritize delivery of the electrical stimulation signal to regions of ventricular organ 104 that the selected operating mode indicates as priority regions.

Processing circuitry 202 may determine the specific values of the parameters of the electrical stimulation signals based at least in part on the detected sensor signals. After determination of the specific values, processing circuitry 202 may transmit instructions to switching circuitry 204 and signal generation circuitry 206 to deliver the electrical stimulation signal to vestibular organ 104. In some examples, based on the detected electrical signals in response to the delivered signal, processing circuitry 202 may adjust one or more parameters of the electrical stimulation signal, e.g., to increase or decrease a rate at which the electrical stimulation signal is delivered to vestibular organ 104. In some examples, processing circuitry 202 may adjust the one or more parameters to adjust a rate at which a target amplitude for the electrical stimulation signal is reached by signal generation circuitry 206 and delivered to vestibular organ 104. In some examples, processing circuitry 202 adjusts the one or more parameters to increase a rate at which electrical charge leaves vestibular organ 104 and/or one or more nerves connected to vestibular organ 104 after IMD 108 ceases delivery of the electrical stimulation signal.

Processing circuitry 202 may, based on the detected electrical signals, adjust the one or more parameters of the electrical stimulation signal to match an electrical charge decay profile of patient 102 to a user-selected decay profile. The user-selected decay profile may be determined by the clinician based on characteristics of patient 102 and/or characteristics of a pool of other patients who are representative of patient 102. In some examples, IMD system 100 may apply one or more machine learning techniques, e.g., via one or more computing systems, devices, and/or cloud computing environments, to determine the user-selected decay profile based at least in part on the characteristics of patient 102 and/or the characteristics of the other patients.

In the one or more machine learning techniques, processing circuitry 202 may retrieved a trained model, and execute the trained model to adjust the one or more parameters. The trained model may be generated based on training datasets that define pre-known user-selected decay profiles for patient characteristics. The training dataset may be used to train a multi-layer neural network, such as by adjusting weights and offsets to nodes of the neural network to minimize a loss function. The above is one example of generating a trained model, and the examples for machine learning techniques are not limited to the above example. There may be various ways to train the neural network.

Processing circuitry 202 may detected changes in the motion data from sensing circuitry 208 and/or one or more of sensor(s) 212. Based on the detected changes, processing circuitry 202 may adjust the one or more parameters of the electrical stimulation signal. Processing circuitry 202 may reference the detected changes to the reference values of the operating states stored in patient behavior module 218 to determine if patient 102 is in different operating state. In some examples, processing circuitry 202 may determine that patient 102 is in a different operating state based on a determination that the detected changes in motion data persists for a threshold period of time. The threshold period of time may be predetermined and may be based on characteristics of patient 102. Based on a determination that patient 102 is in a different operating state, processing circuitry 202 may retrieve and execute instructions for the corresponding new operating mode from operating mode module 220. Processing circuitry 202 may adjust the one or more parameters of the electrical stimulation signal, begin transmission of the electrical stimulation signal, or cease transmission of the electrical stimulation signal based on the new operating mode.

Memory 216 may include one or more modules including patient behavior module 218 and operating mode module 220. Patient behavior module 218 may store, for each possible operating state of patient 102 (e.g., rest, light activity, heavy activity (e.g., exercise, running), vehicle use (e.g., automobile, plane, or train)), reference values (e.g., reference ranges) for each of the one or more metrics determined by processing circuitry 202. In some examples, the reference values for each of the one or more metrics may be based on characteristics of patient 102. In some examples, the references values may be based on characteristics of other patients who are representative of patient 102 (e.g., with similar age, weight, medical conditions, fitness, or the like).

Operating mode module 220 may store instructions for IMD 108 to transmit electrical stimulation signals in a particular manner (e.g., within a range of pre-defined frequencies, within a range of pre-defined amplitudes, within priority or avoidance of particular regions of vestibular organ 104, or the like). In some examples, each of the operating states stored in patient behavior module 218 may correspond to one or more operating modes stored in operating mode module 220. In some examples, multiple operating states stored in patient behavior module 218 may correspond to a single operating mode stored in operating mode module 220.

Communications circuitry 210 supports wireless communication between device 104 and an external programmer or another computing device of IMD system 100. In some examples, communications circuitry 210 may support wireless communication between two or more IMDs (e.g., between the first IMD and the second IMD). Communications circuitry 210 may accomplish communication by radiofrequency (RF) communication techniques, e.g., via an antenna (now shown).

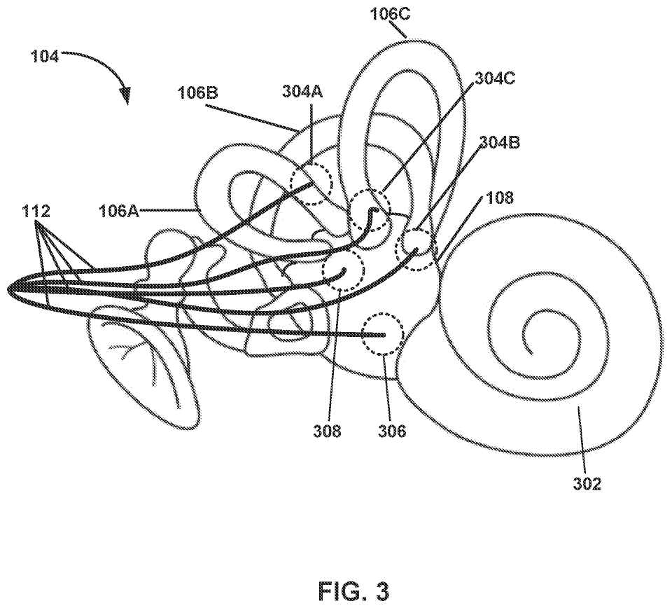

FIG. 3 is a conceptual diagram illustrating connections of electrical leads 112 of IMD system 100 of FIG. 1 to vestibular organ 104. Vestibular organ 104 includes vestibule 108 and semicircular canals 106A-C (collectively referred to as “semicircular canals 106”). Vestibular organ 104 is connected to and located behind cochlea 302.

Vestibule 108 includes otolith organs saccule 306 and utricle 308. Saccule 306 and utricle 308 are fluid-filled cavities containing hair cells and forming parts of the membranous labyrinth. Saccule 306 detects linear vertical movement of the head of patient 102 and translates the detected movement to neural input. Utricle 308 detects linear horizontal movement of the head of patient 102 and translates the detected movement to neural input. Semicircular canals 106 are three fluid-filled canals attached to vestibule at bases of each canal and include horizontal semicircular canal 106A, posterior semicircular canal 106B, and superior semicircular canal 106C (also referred to as “anterior semicircular canal 106C”). Horizontal semicircular canal 106A detects rotation of the head of patient 102 in the transverse plane of patient 102 (e.g., when patient 102 turns their head from side to side). Posterior semicircular canal 106B detects rotation of the head of patient 102 in the coronal plane (e.g., such as when patient 102 rotates their head towards their shoulder). Superior semicircular canal 106C detects rotation of the head in the sagittal plane of patient 102 (e.g., when patient 102 is nodding their head).

As illustrated in FIG. 3, electrical leads 112 may be connected to semicircular canals 106 at target locations 304 A-C, (collectively referred to as “target locations 304”). Each of target locations 304 may be at a base of the corresponding semicircular canal 106 (e.g., as illustrated in FIG. 3, or may be in any other region along the corresponding semicircular canal 106. Electrical leads 112 may also be connected to saccule 306 and utricle 308, e.g., at an outer surface of vestibule 108, as shown in FIG. 3. In some examples, depending on the impairment of vestibular organ 104 of patient 102, electrical leads 112 may be connected to some or all of saccule 306, utricle 308, or semicircular canals 106.

FIG. 4A is a conceptual diagram illustrating an example location for the IMDs (e.g., first IMD 402 and second IMD 404) of IMD system 100 of FIG. 1. FIG. 4B is a conceptual diagram illustrating another example location of IMD 108 of IMD system 100 of FIG. 1. As illustrated in FIGS. 4A and 4B, at least one IMD (e.g., second IMD 404, IMD 108 may be implanted subcutaneously within patient 102. Electrical lead(s) 112 of IMD system 100 may be connected to vestibular organ 104 via channel 408 created within temporal bone 405 of patient 102.

First IMD 402 may be disposed within a cranial cavity of patient 102 and may be secured to temporal bone 405 by one or more mounting devices 406 (e.g., brackets). Electrical lead(s) 112 may extend from first IMD 402 and connect regions of vestibular organ 104 via channel 408. First IMD 402 may receive instructions from second IMD 404 (e.g., via communications circuitry 210), generate electrical stimulation signals based on the received instructions, and transmit the electrical stimulation signals to regions of vestibular organ 104 in accordance with the received instructions.

Second IMD 404 may be disposed in a different location within the head of patient 102 than first IMD 402. In some examples, as illustrated in FIG. 4A, second IMD 404 may be disposed outside of the cranial cavity. Second IMD 404 may be disposed at the base of the neck and close to the spine of patient 102). Placing second IMD 404 outside of the cranial cavity of patient 102 may allow easier access to second IMD 404. Second IMD 404 may house processing circuitry 202, sensing circuitry 208, a power source (e.g., power source 214, communications circuitry 210. memory 216, and one or more of sensor(s) 212 (e.g., an accelerometer, a gyroscope, or the like). Second IMD 404 may be disposed close to the centerline of the head of patient 102 to enable more accurate detections of movements of the head by the one or more sensors 212. Based on the detected movements, second IMD 404 may select an appropriate operating state of patient 102, select a corresponding operating mode from memory 216, determine parameters of an electrical stimulation signal, and transmit instructions to first IMD 402 to transmit the electrical stimulation signal to vestibular organ 104 (e.g., in accordance with the one or more examples described herein.).

FIG. 4B illustrates an example IMD system 100 with a single IMD 108. IMD 108 may be disposed within a same or similar location within head of patient 102 as second IMD 402 and may include the circuitry and components of IMD 108 of FIG. 2.

As illustrated in FIGS. 4A and 4B, an IMD(e.g., second IMD 404, IMD 108, or the like) may include one or more electrical leads 410 extending out of second IMD 404. Electrical leads 410 may be connected to one or more implantable sensors disposed within the neck of the patient and may transmit any electrical signals detected by the implantable sensors to second IMD 404 (e.g., to processing circuitry 202 and/or sensing circuitry 208.

Electrical leads 112 may be advanced through one or more tunnels in temporal bone 405 and into channel 408, which is illustrated and described in greater detail in FIG. During implantation of IMD system 100, the clinician may form tunnels, e.g., as illustrated in FIGS. 4A and 4B to connect IMDs to vestibular organ 104. In some examples, as illustrated in FIG. 4B., the tunnel containing electrical lead(s) 112 may extend behind the middle and inner ear of patient 102 and connect with vestibular organs 104.

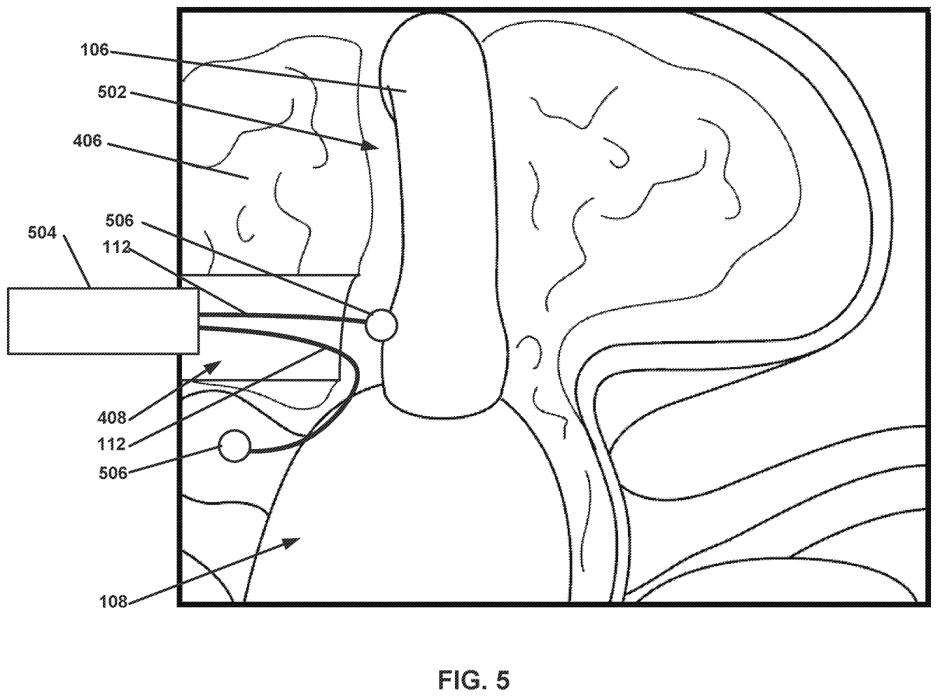

FIG. 5 is a conceptual diagram illustrating electrical lead(s) 112 of FIG. 1 disposed within channel 408 in temporal bone 406 of patient 102. Temporal bone 406 may envelop vestibular organ 104 (e.g., semicircular canals 106 and vestibule 108). Temporal bone 406 may be separate from the tissue of vestibular organ 104 by recess 502. The clinician may create a single channel 408 to access recess 502 and position electrical lead(s) 112 on regions of vestibular organ 104 through channel 408.

During implantation of IMD system 100, the clinician may form channel 408 through temporal bone 406, e.g., via a drill. As illustrated in FIG. 5, channel 408 may access recess 502 at the base of semicircular canals 106 (e.g., a region of vestibular organ 104 wherein semicircular canals 106 connect to vestibule 108. Channel 408 may extend within temporal bone 406 into recess 502 around space 502 around semicircular canals 106. The clinician may advance a catheter 504 containing electrical lead(s) 112 disposed within an inner lumen of catheter 504 into channel 408. The clinician may then advance electrical lead(s) 112 out of catheter 504 and into recess 502 and affix electrical lead(s) 112 to regions vestibular organ 104 (e.g., to semicircular canals 106, to vestibule 108) through channel 408 without creating additional channels. Electrical lead(s) 112 may exit channel 408 and navigate to the corresponding regions of vestibular organ 104 through recess 502. The clinician may manipulate electrical lead(s) 112 via a guide member (e.g., a stylet disposed within the inner lumen of catheter 504). The guide member may create torsion within electrical lead(s) 112, e.g., to better adhere the distal ends of electrical lead(s) 112 to the outer surface of vestibular organ 104.

Each electrical lead 112 may include an attachment device 506 disposed on the distal end of electrical lead 112. Attachment device 506 may affix the distal end of electrical lead 112 (e.g., electrode 222 of electrical lead 112) to a user-selected region of vestibular organ 104. In some examples, attachment device 506 may penetrate the outer surface of vestibular organ 104. In some examples, attachment device 506 may affix to the outer surface of vestibular organ 104 without penetrating the outer surface. For example, the attachment device 506 may include a plurality of protrusions extending from the distal end of electrical lead 112, the plurality of protrusions configured to adhere to the outer surface of electrical lead 112. In some examples, the attachment device 506 may be an adhesive material attached to the distal end of electrode 112 such as, but is not limited to, hydroxyapatite.

In some examples, hydroxyapatite may be disposed on the distal end of electrical lead 112. The clinician may advance electrical lead 112 with the disposed hydroxyapatite through channel 408 and place the distal end of electrical lead 112 on the outer surface of vestibular organ 104. The clinician may manipulate the position of the distal end of electrical lead 112 before the hydroxyapatite can harden. Once the clinician determines that the placement is correct, the clinician may let the hydroxyapatite harden and secure the distal end of electrical lead 112 to vestibular organ 104.

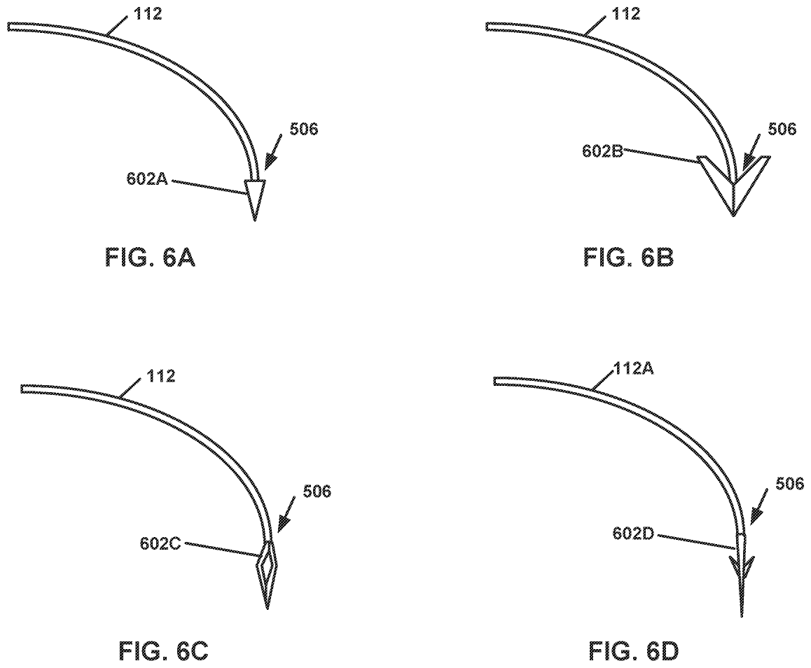

FIGS. 6A-F illustrate example attachment devices 506 of IMD system 100 of FIG. 1. FIGS. 6A-6F illustrate attachment devices 506 having fixation members 602A-F (collectively referred to as “fixation members 602”). FIGS. 6A-6D illustrate attachment devices 506 having fixation members 602A-D that may penetrate the outer surface of vestibular organ 104 to connect electrical lead(s) 112 to vestibular organ 104. FIGS. 6E and 6F illustrate attachment devices 506 having fixation members 602E-F having protrusions 604 configured to affix electrical lead(s) 112 to vestibular organ 104 without penetrating vestibular organ 104 The example attachment devices 506 of FIGS. 6A-F may be permanently connected to electrical lead(s) 112. In other examples, such as with the use of adhesive materials as described above, attachment device 506 may be removably connected to electrical lead(s) 112. In some examples, attachment devices 506 (e.g., fixation members 602) may be electrodes 222.

Fixation member 602A has a pyramidal shape and may penetrate the outer surface of a region of vestibular organ 104 to connect electrical lead 112 to vestibular organ 104. Fixation member 602B includes a plurality of flanges extending radially outwards from electrical lead 112. A distal tip of fixation member 602B may penetrate the outer surface of vestibular organ 104 and the plurality of flanges may hook into tissue of vestibular organ 104 to secure fixation member 602B within vestibular organ 104. Fixation member 602C may have a diamond shape and may penetrate the outer surface of vestibular organ 104 while one or more edges of fixation member 602C may hook onto tissue of vestibular organ 104. Fixation member 602D includes a sharp distal end configured to penetrate the outer tissue of vestibular organ 104 and a plurality of tines extending proximally and radially outwards. The tines may hook into tissue of vestibular organ 104 to affix fixation member 602D within the tissue.

Fixation member 602E and fixation member 602F may include a flat surface having a plurality of protrusions 604. Protrusions 604 may adhere to the outer surface of vestibular organ 104 without penetrating vestibular organ 104 via friction, adhesives or other means. In some examples, the clinician may affix an adherence tool (not pictured) onto the outer surface of vestibular organ 104 and fixation member 602E or fixation member 602F may be secured to the adherence tool via protrusions 604 through application of a first force in a first direction. Fixation members 602E-F may resist fixation to adherence tool with applications of force in directions other than the in the first direction. Fixation member 620E may then be released from the second fixation member via an application of a second force in a second direction, wherein the second direction is in the opposite direction as the first force. Adherence tool and/or protrusions 604 may include a metallic material and/or a biocompatible polymer (e.g., a corrugated polymer).

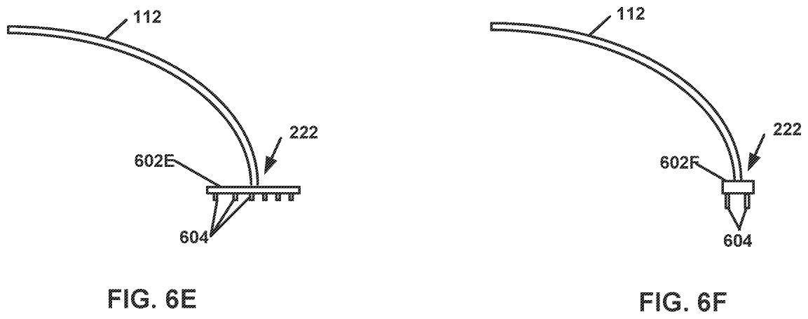

FIG. 7 is a conceptual diagram illustrating example stimulation modes of the system of FIG. 1. Each of operating modes 710 A-D (collectively referred to as “operating modes 812”) may include one or more metrics (e.g., angular shift frequency 702, evoked electrical signal 704) used by IMD 108 to determine an operating state 706 of patient 102. Each of operating modes 710 may include a stimulation pattern 708 corresponding to the operating state 706. In some examples, each of operating modes 710 may have a same nomenclature and/or label as the corresponding operating state 706. Operating state 706 may include, but are not limited to, “vehicle” (e.g., patient 102 is traveling within a vehicle), “dynamic exercise”, “sleep”, and “moderate exercise” (e.g., walking, jogging). While FIG. 7 illustrates four operating modes 710A-D, other example IMD systems 110 may have three or fewer or five or more operating modes 710.

For example, IMD system 100 may deliver electrical stimulation signals to vestibular system 104 in accordance with operating mode 710A when patient 102 is in a vehicle (e.g., an automobile). While patient 102 is in a vehicle, patient may experience high angular shift frequency, e.g., due to patient 102 rotating their head to look at road conditions, traffic, the rear view mirrors, or the like. At the same time, IMD system 100 may detect low evoked electrical signals due to the relatively relaxed and sedentary nature of operating or using a vehicle, e.g., relative to heavy physical activity. Based on the detected angular shift frequency 702 and evoked electrical signal 704, IMD system 100 may determine that operating state 706 of patient 102 is “vehicle” and may favor delivery of horizontal semicircular canal and utricle stimulation, e.g., to ensure that patient 102 is not impaired when the head of patient 102 experiences horizontal movement and/or rotation about the transverse plane.

When operating mode 710 (e.g., operating mode 710B) includes stimulation pattern 708 of “full activation”, IMD system 100 may deliver electrical stimulation signals to vestibular organ 104 to the full capabilities of IMD system 100, if necessary. When operating mode 710 (e.g., operating mode 710C) includes stimulation pattern 708 of “limited activation,” operating mode 710 may limit one or more parameters of the electrical stimulation signal (e.g., maximum amplitude, maximum frequency, maximum number of electrical stimulation signals per a predetermined period of time, maximum number of regions of vestibular organ 104 to be stimulated at any given time, or the like). Operating mode 710 may limit the one or more parameters of the electrical stimulation signal, e.g., to reduce power usage during periods of time when patient 102 requires limited stimulation (e.g., when patient 102 is asleep).

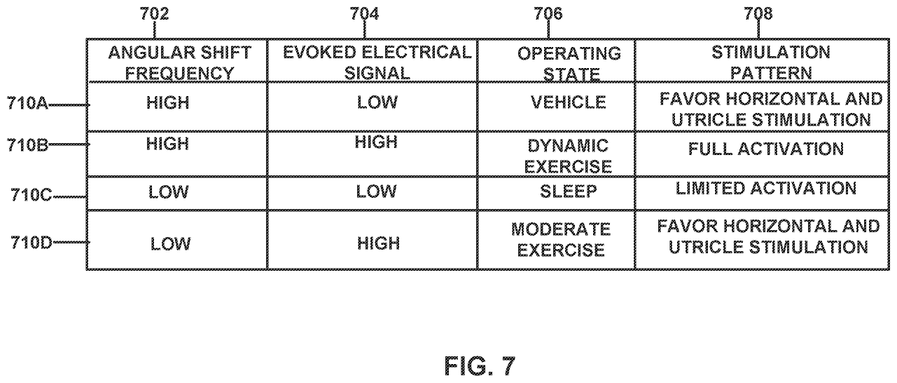

FIG. 8 is a conceptual diagram illustrating other example stimulation modes of the system of FIG. 1. Compared to operating modes 710, operating modes 808A-E (collectively referred to as “operating modes 808”) may include additional metrics including acceleration rate 802. The use of additional metrics may allow IMD system 100 to better distinguish patient behavior and to select an appropriate operating state 804 form a plurality of operating states 804. For example, IMD system 100 may determine that patient 102 is in a vehicle and may select the type of vehicle patient 102 is in as operating state 804 (e.g., “automobile” in operating state 808A, “plane/train” in operating state 808D). Operating modes 808 may also include an increased number of stimulation patterns 806 that correspond to the increased range of available operating states 804.

For example, operating modes 808C and 808D may both have similar angular shift frequencies 702 (“LOW”) and evoked electrical signals 704 (“HIGH”). Operating mode 808D may have a “HIGH” acceleration rate 802 due to movement of a transport (e.g., an airplane, a train, or the like) while patient 102 is aboard the transport while operating mode 808C may have a “LOW” acceleration rate 802 while patient 102 is asleep (e.g., within a building). Based at least in part on acceleration rate 802, IMD system 100 may correspond a scenario with the metrics of operating mode 808C with operating state 804 of “sleep” and may limit activation of IMD 108, e.g., due to a lack of necessary stimulation while patient 102 is asleep. IMD system 100 may also correspond a scenario with the metrics of operating mode 808D with operating state “804” of “plane/train” and may instruct IMD 108 to favor utricle stimulation, e.g., due to the horizontal movement of patient 102.

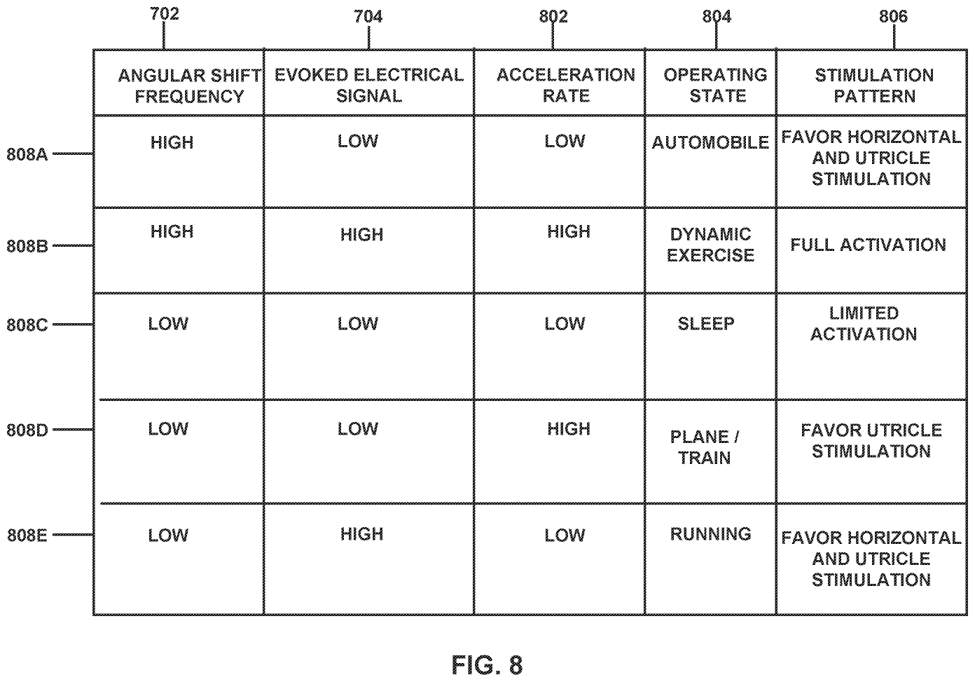

FIG. 9 is a conceptual diagram illustrating an example user interface (UI) displaying an example process of adjusting electrical stimulation signals transmitted by the system of FIG. 1. IMD system 100 may perform the example process illustrated and described in FIG. 9 based at least in part on motion data (e.g., from sensor(s) 212).

For a given threshold time 901 (e.g., a predetermined period of time in minutes, hours, days, or the like), IMD system 100 may deliver an electrical stimulation signal 904 with a varying stimulation amplitudes 910A (e.g., in a bell curve as illustrated in FIG. 9) to one or more targeted region 910B. In some examples, IMD system 100 may deliver electrical stimulation signal 904 to one or more regions of vestibular organ 104 (e.g., to saccule 306, to utricle 308, and/or to one or more of semicircular canals 106) simultaneous. In some examples, as illustrated in FIG. 9, IMD system 100 may deliver electrical stimulation signal 904 to one or more regions consecutively such that electrical stimulation signal 904 to the different regions do not overlap.

IMD system 100 may adjust the timing of electrical stimulation signal 904 (e.g., stimulation amplitude 910A and/or targeted region 910B) to correspond stimulation of a target region 910B with device inputs 902 (e.g., from sensor(s) 212 of IMD 108). Sensors 908A-C (collectively referred to as “sensors 908”) may each detect a different movement of the head of patient 102 (e.g., vertical movement, horizontal movement, and/or rotation about one or more planes). IMD system 100 may adjust the timing of electrical stimulation signal 904 to deliver electrical stimulation signal 904 to targeted region 910B of vestibular organ 104 at the same time targeted region 910B is expected to detect movement of the head of patient 102 and translate the detected movement into neural inputs. IMD system 100 may adjust the rate at which stimulation amplitude 910A increases and/or decreases to match electricals stimulation signal 904 to the timing of device inputs 902.

In some examples, IMD system 100 may adjust the frequency of electrical stimulation signal 904 (alternatively referred to herein as a “pulse frequency” of electrical stimulation signal 904) to correspond stimulation of target region 910B with device inputs 902. IMD system 100 may adjust the pulse frequency of electrical stimulation signal 904 to deliver electrical stimulation signal 904 to targeted region 910B of vestibular organ 104 at the same time targeted region 910B is expected to detect movement of the head of patient 102, and translate the detected movement into neural inputs. IMD system 100 may increase or decrease the pulse frequency of electrical stimulation signal 904.

IMD system 100 may also determine stimulation calibration 906 based on detected electrical signals (e.g., from the implanted sensors) and adjust parameters of stimulation signal 904 based at least in part on the detected electrical signals. As illustrated in FIG. 9, stimulation calibration 906 includes nerve stimulation and signal saturation status 912 (hereinafter referred to as “nerve status 912”), other metrics and indications may be used in other examples.

IMD system 100 may detect, as a part of nerve status 912, increasing saturation levels, decaying saturation levels, and/or lack of electrical charge in one or more nerves of patient 102. IMD system 100 may adjust the parameters of electrical stimulation signal 904 to ensure that nerves within target region 910B is clear of electrical charge at the end of stimulation to targeted region 910B, e.g., to prevent unintended stimulation. In some examples, IMD system 100 may adjust the parameters of electrical stimulation signal 904 to ensure that a nerve in targeted region 910B is cleared of electrical charge prior to the next electrical stimulation signal 904 to targeted region 910B, e.g., to prevent unintended and/or excessive stimulation.

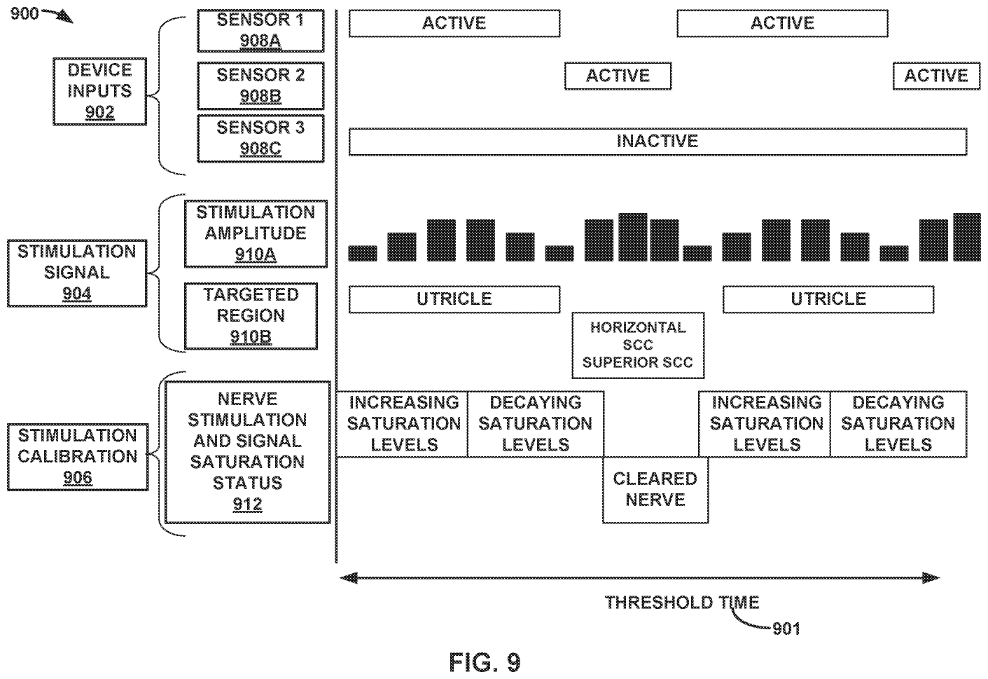

FIG. 10 is a flow diagram illustrating an example process of delivering electrical stimulation signals to vestibular organ 104 of patient 102. The example process illustrated in FIG. 10 may be performed using a medical device system (e.g., IMD system 100) or an implantable medical device (e.g., IMD 108, first IMD 402, second IMD 404, or the like). In some examples, the techniques and processes discussed in this disclosure may include additional or fewer steps than the example process of FIG. 10. In some examples, the example process may be performed using an IMD with a different design that IMD 108, first IMD 402, or second IMD 404.

IMD system 100 may detect movement of patient 102 (1002). IMD system 100 may use sensor(s) 212 connected to one or more IMDs of IMD system 100 to detect movement and/or rotation of the head of patient 102. Sensor(s) 212 include accelerometers, gyroscopes, implanted sensors detecting electrical signals (e.g., evoked electrical signals, myogenic potentials, etc.) in tissue of patient 102, or other similar motion-detecting sensors. In some examples, IMD system 100 may detect, by a gyroscope of IMD system 100, movements of the head. In some examples, IMD system 100 detects, by an implantable sensor of IMD system 100, the electrical signals in tissue of patient 102. In some examples, IMD system 100 detects, by an accelerometer of IMD system 100, changes in acceleration of the head.

IMD system 100 may determine an operating mode (e.g., operating modes 710 as illustrated in FIG. 7 , operating modes 808 as illustrated in FIG. 8) based on the detected movement (1004). IMD system 100 may determine, by processing circuitry of IMD system 100 and based on the detected movement of head, an angular shift frequency of the head. IMD system 100 may determine, by the processing circuitry of IMD system 100 and based on the detected electrical signals, an evoked electrical signal in the tissue of patient 102 in response to the movements of the head of patient 102. IMD system 100 may determine, by the processing circuitry and based on the detected changes in acceleration, an acceleration rate of the head of patient 102. IMD system 100 may select, by the processing circuitry and based on determined characteristics of patient 102 comprising the angular shift frequency, the evoked electrical signal, and/or the acceleration rate, an operating mode for IMD 108 of IMD system 100 from a plurality of operating modes stored in memory of IMD system 100.

In some examples, IMD system 100 may determine an operating state of patient 102 based on the detected movement by comparing the detected metrics to reference values and/or ranges for operating states stored within IMD system 100. IMD system 100 may select a corresponding operating mode based on the determined operating state. Operating modes may include instructions including limits (e.g., amplitude and/or frequency limits) to an electrical stimulation signal, particular regions of vestibular organ 104 to prioritize or avoid stimulation, and more.

IMD system 100 may deliver electrical stimulation to vestibular organ 104 of patient 102 based on the determined operating mode (1006). IMD system 100 may determine, based on the detected movement metrics, one or more parameters (e.g., amplitude, frequency, targeted region) to the electrical stimulation signals. IMD system 100 may ensure that the determined electrical stimulation signals satisfies the limitations and/or conditions of the determined operating mode. Based on a determination that the electrical stimulation signals are satisfactory, IMD system 100 may generate and transmit the electrical stimulation signals to vestibular organ 104 of patient 102 via electrical lead(s) 112. In some examples, IMD system 100 may repeat steps 1002-1006 of the example process and may adjust the operating mode and parameters of the electrical stimulation signal (e.g., from a first operating mode to a second operating mode) based on changes in movement of the head of patient 102. For example, IMD system 100 may determine, based on changes in at least one of the determined characteristics, a change in action by patient 102. IMD system 100 may then select, by the processing circuitry and based at least in part on the determined change in action, a second operating mode from the plurality of operating modes stored in memory of IMD system 100 and transmit, by IMD 108, a second electrical stimulation signal to vestibular organ 104 of patient 102 based on the second operating mode. In some examples, IMD system 100 determines whether a duration of the determined change in action of patient 102 satisfies a threshold condition (e.g., a threshold period of time) and select the second operating mode based on the determination that the threshold condition is satisfied.

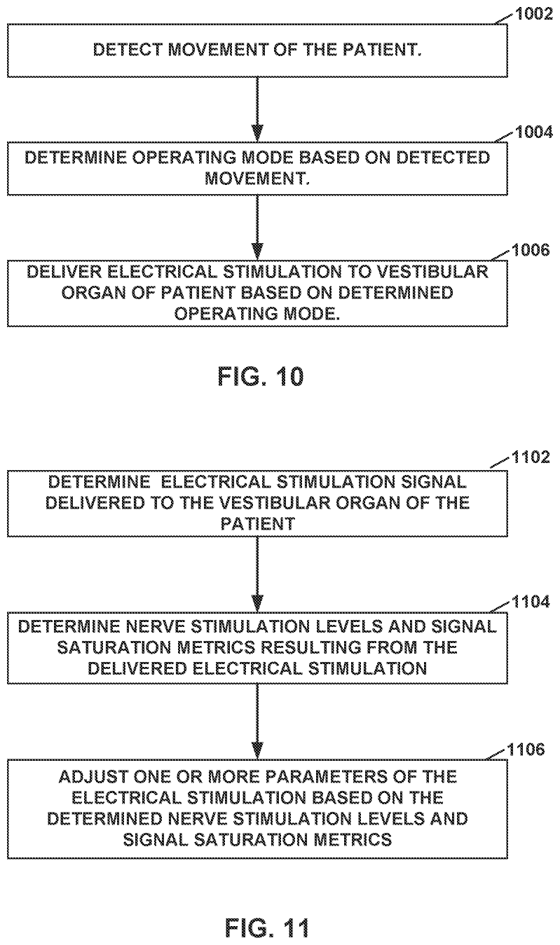

FIG. 11 is a flow diagram illustrating an example process of adjusting electrical stimulation signals based on patient response. Since patient characteristics vary from patient to patient, IMD system 100 may determine actual effects of delivered electrical stimulation signal on patient 102 and adjust the parameters of the electrical stimulation signal to increase efficacy of the stimulation.

IMD system 100 may determine that an electrical stimulation signal is delivered to vestibular organ 104 of patient 102 (1102). IMD system 100 may transmit, via IMD 108 of IMD system 100, the electrical stimulation signal to one or more regions of vestibular organ 104 of patient 102. IMD system 100 may determine delivery of the electrical stimulation signal based at least in part on changes and/or presence of electrical charge in one or more nerves of patient 102. In some examples IMD system 100 may detect, via an implantable sensor of IMD system 100, an electrical signal or patient 102 in response to the transmitted electrical stimulation signal and determine, by processing circuitry and based on the detected electrical signal, that IMD 108 is transmitting the electrical stimulation signal to a nerve at a first rate of change of amplitude of the electrical stimulation signal. IMD system 100 may determine nerve stimulation levels and signal saturation metrics resulting from the delivered electrical stimulation (1104). Signal saturation metrics may include signal saturation status, signal saturation degree, or signal saturation decay. Nerve stimulation levels may correspond to a rate at which the electrical stimulation signal reaches a target amplitude.

IMD system 100 may adjust one or more parameters of the electrical stimulation signal based on the determined nerve stimulation levels and signal saturation metrics (1106). IMD system 100 may determine adjust amplitude, frequency, and/or other parameters of electrical stimulation signal to correspond electrical stimulation signal with characteristics of patient 102 (e.g., a rate at which electrical charge decays in the nerve, a rate at which electrical charge increases in the nerves, timing of movements detected by sensors, timing of stimulation to different regions of vestibular organ 104, or the like). IMD system 100 may determine that the first rate of change of the amplitude satisfies a threshold condition and adjust, based on the determination, one or more parameters of the electrical stimulation signal to cause IMD 108 to transmit the electrical stimulation signal to the nerve at a second rate of change of the amplitude.

In some examples, IMD system 100 determines, based on the detected electrical signal or the one or more parameters of the electrical stimulation signal, one or more signal saturation metrics. IMD system 100 may determine whether at least one of the one or more signal saturation metrics satisfies a signal saturation threshold condition (e.g., a rate of decay, a minimum electrical charge in a nerve, or the like). IMD system 100 may adjust, based on the determination that at least one of the one or more signal saturation metrics satisfies the corresponding signal saturation threshold condition, the one or more parameters of the electrical stimulation signal to cause IMD 108 to transmit the electrical stimulation signal to the nerve at the second rate of change of the amplitude.

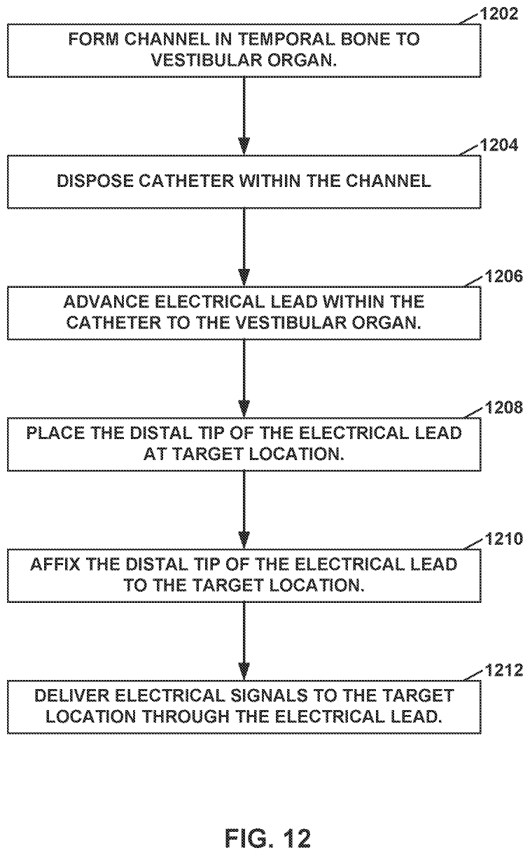

FIG. 12 is a flow diagram illustrating an example process of implanting the IMD system 100 of FIG. 1. The clinician may form a channel (e.g., channel 408) in temporal bone 406 of patient 102 to vestibular organ 104 (e.g., to recess 502 surrounding vestibular organ 104) (1202). The clinician may form channel 408. from within the cranial cavity of patient 102 and/or from below temporal bone 406, around the middle and inner ear of patient 102, and towards vestibular organ 104. The clinician may form channel 408 using one or more drilling apparatuses and/or devices.

The clinician may dispose a catheter (e.g., catheter 504) within channel 408 (1204). The clinician may navigate catheter 504 within channel 408 until a distal end of catheter 504 nears or enters recess 502 around vestibular organ 104. The clinician may advance electrical lead 112 from within catheter 504 to vestibular organ 104 (1206). In some examples, the clinician may advance electrical lead 112 out of catheter 504 and place electrical lead 112 directly on outer surface of vestibular organ 104 (e.g., at user-selected regions of vestibular organ 104). In some examples, the clinician may advance electrical lead 112 into recess 502 and navigate electrical lead 112 within recess 502 to the user-selected regions of vestibular organ 104. The clinician may navigate electrical lead 112 using one or more inner members (e.g., stylets or other guide members) connected to proximal ends of electrical lead 112.

The clinician may place the distal tip of electrical lead 112 at target locations on vestibular organ 104 (1208). The target location may be the user-selected region of vestibular organ 104. The clinician may then affix the distal tip of electrical lead 112 to the target location (1210). The clinician may affix the distal tip of electrical lead 112 via an attachment device (e.g., attachment device 506) disposed on a distal end of electrical lead 112. Attachment device 506 may affix to vestibular organ 104 via penetration, adhesion (e.g., using an adhesive, using an adhesion tool disposed on vestibular organ 104), or the like. The clinician may then deliver electrical signals (e.g., electrical stimulation signals) to the target location through electrical lead 112 (1212).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

In addition, it should be noted that system described herein may not be limited to treatment of a human patient. In alternative examples, the system may be implemented in non-human patients, e.g., primates, canines, equines, pigs, and felines. These other animals may undergo clinical or research therapies that may benefit from the subject matter of this disclosure.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Example 1A: a vestibular implant system comprising: a memory; a gyroscope configured to detect movements of a head of a patient; an implantable sensor configured to detect electrical signals in tissue of the patient; a vestibular implant; and processing circuitry coupled to the memory, the processing circuitry configured to: determine, based on the detected movements of the head, an angular shift frequency of the head; determine, based on the detected electrical signals, an evoked electrical signal, sensed by the implantable sensor, in the tissue of the patient in response to the movements of the head; and select, based on determined characteristics comprising the angular shift frequency and the evoked electrical signal, an operating mode for the vestibular implant from a plurality of operating modes stored in the memory, wherein the vestibular implant is configured to transmit an electrical stimulation signal to one or more regions of a vestibular organ based on the selected operating mode.

Example 2A: the vestibular implant system of example 1A, further comprising: an accelerometer configured to detect changes in acceleration of the head of the patient, wherein the processing circuitry is further configured to: determine, based on the detected acceleration, an acceleration rate of the head of the patient, and wherein the determined characteristics further comprises the acceleration rate of the head.

Example 3A: the vestibular implant system of any of examples 1A and 2A, wherein to select the operating mode for the vestibular implant, the processing circuitry is further configured to: determine, based on the determined characteristics of the patient, an action of the patient; and select, based on the determined action of the patient, the operating mode from the plurality of operating modes, the operating mode comprising parameters of the electrical stimulation signal that, when transmitted to the one or more regions of the vestibular organ, cause the patient to maintain balance while performing the action.

Example 4A: the vestibular implant system of example 3A, wherein the processing circuitry is further configured to: determine, based on changes in at least one of the determined characteristics, a change in the action by the patient; select, based at least in part on the determined change in action, a second operating mode from the plurality of operating modes; and wherein the vestibular implant is further configured to: transmit a second electrical stimulation signal to the one or more regions of the vestibular organ of the patient based on the second operating mode.

Example 5A: the vestibular implant system of example 4A, wherein the processing circuitry is further configured to: determine whether a duration of the determined change in action of the patient satisfies a threshold condition; and select, based on the satisfaction of the threshold condition, the second operation mode.

Example 6A: the vestibular implant system of any of examples 3A-5A, wherein at least one operating mode of the plurality of operating modes comprises parameters of the electrical stimulation signal that, when transmitted to the one or more regions of the vestibular organ, cause the patient to maintain balance while the patient is immobile.

Example 7A: the vestibular implant system of any of examples 3A-6A, wherein at least one operating mode of the plurality of operating modes comprises parameters of the electrical stimulation signal that, when transmitted to the one or more regions of the vestibular organ, cause the patient to maintain balance while the patient is in a moving vehicle.

Example 8A: the vestibular implant system of any of examples 3A-7A, wherein at least one operating mode of the plurality of operating modes comprises parameters of the electrical stimulation signal that, when transmitted to the one or more regions of the vestibular organ, cause the patient to maintain balance while the patient is in motion.

Example 9A: the vestibular implant system of any of examples 1A-8A, wherein the one or more regions of the vestibular organ comprises one or more semicircular canals or one or more otolith organs.

Example 10A: the vestibular implant system of example 9A, wherein the operating mode is configured to prioritize stimulation of at least one of the one or more semicircular canals or at least one of the one or more otolith organs over another region of the one or more regions of the vestibular organ.

Example 11A: the vestibular implant system of any of examples 1A-10A, wherein the implantable sensor is configured to detected electrical signals in one or more muscles in a neck of the patient corresponding to the movements of the head of the patient.

Example 12A: the vestibular implant system of any of examples 1A-11A, wherein the evoked electrical signal comprises cervical compound muscle action potential.

Example 13A: the vestibular implant system of any of examples 1A-12A, wherein each of the plurality of operating modes comprises parameters of a corresponding electrical stimulation signal.

Example 14A: the vestibular implant system of example 13A, wherein the parameters of the corresponding electrical stimulation signal comprise an amplitude and a frequency of the corresponding electrical stimulation signal.

Example 15A: the vestibular implant system of any of examples 1A-14A, further comprising one or more motion sensors, the one or more motion sensors configured to detect motion data corresponding to movement of the patient, and wherein the processing circuitry is further configured to determine, based on the detected motion data, at least one of a gait or a posture of the patient, wherein the determined characteristics of the patient further comprises the at least one of the gait or the posture of the patient.

Example 16A: a method comprising: detecting, by a gyroscope of a vestibular implant system, movements of a head of a patient; detecting, by an implantable sensor of the vestibular implant system, electrical signals in tissue of the patient; determining, by processing circuitry of the vestibular implant system and based on the detected movements of the head, an angular shift frequency of the head; determining, by the processing circuitry and based on the detected electrical signals, an evoked electrical signal in the tissue of the patient in response to the movements of the head; selecting, by the processing circuitry and based on determined characteristics of the patient comprising the angular shift frequency and the evoked electrical signal, an operating mode for a vestibular implant of the vestibular implant system from a plurality of operating modes stored in memory of the vestibular implant system; and transmitting, by the vestibular implant of the vestibular implant system, an electrical stimulation signal to the one or more regions of the vestibular organ based on the selected operating mode.

Example 17A: the method of example 16A, further comprising: detecting, by an accelerometer of the vestibular implant system, changes in acceleration of the head; and determining, by the processing circuitry and based on the detected changes in acceleration, an acceleration rate of the head of the patient, wherein the determined characteristics of the patient further comprise the acceleration rate of the head of the patient.

Example 18A: the method of any of examples 16A and 17A, wherein selecting the operation mode for the vestibular implant further comprises: determining, by the processing circuitry and based on the determined characteristics of the patient, an action of the patient; and selecting, by the processing circuitry and based on the determined action, the operating mode from the plurality of operating modes, the operating mode comprising parameters of the electrical stimulation signal that, when transmitted to the one or more regions of the vestibular organ, cause the patient to maintain balance while performing the action.

Example 19A: the method of example 18A, further comprising: determining, by the processing circuitry and based on changes in at least one of the determined characteristics, a change in action by the patient; selecting by the processing circuitry and based at least in part on the determined change in action, a second operating mode from the plurality of operating modes; and transmitting, by the vestibular implant, a second electrical stimulation signal to the one or more regions of the vestibular organ of the patient based on the second operating mode.

Example 20A: the method of example 19A, further comprising: determining, by the processing circuitry, whether a duration of the determined change in action of the patient satisfies a threshold condition; and selecting, by the processing circuitry, the second operation mode based on the determined satisfaction of the threshold condition.

Example 21A: the method of any of examples 16A-20A, wherein at least one operating mode of the plurality of operating modes comprises parameters of the electrical stimulation signal that, when transmitted to the one or more regions of the vestibular organ, cause the patient to maintain balance while the patient is substantially immobile.

Example 22A: the method of any of examples 16A-21A, wherein at least one operating mode of the plurality of operating modes comprises parameters of the electrical stimulation signal that, when transmitted to the one or more regions of the vestibular organ, cause the patient to maintain balance while the patient is in a moving vehicle.

Example 23A: the method of any of examples 16A-22A, wherein at least one operating mode of the plurality of operating modes comprises parameters of the electrical stimulation signal that, when transmitted to the one or more regions of the vestibular organ, cause the patient to maintain balance while the patient is in motion.

Example 24A: the method of any of examples 16A-23A, wherein the one or more regions of the vestibular organ comprises one or more semicircular canals or one or more otolith organs.

Example 25A: the method of example 24A, wherein the operating mode is configured to prioritize stimulation of at least one of the one or more semicircular canals or at least one of the one or more otolith organs over another region of the vestibular organ.

Example 26A: the method of any of examples 16A-25A, wherein detecting the electrical signals in the tissue of the patient comprises detecting, by the implantable sensor, the electrical stimulation signals in one or more muscles in a neck of the patient corresponding to the movements of the head of the patient.

Example 27A: the method of any of examples 16A-26A, wherein the evoked electrical signal comprises cervical compound muscle action potential.

Example 28A: the method of any of examples 16A-27A, wherein each of the plurality of operating modes comprises parameters of a corresponding electrical stimulation signal.

Example 29A: the method of example 28A, wherein the parameters of the corresponding electrical stimulation signal comprises an amplitude and a frequency of the corresponding electrical stimulation signal.

Example 30A: the method of any of examples 16A-29A, further comprising: detecting, by one or more motions sensor of the vestibular implant system, motion data corresponding to movement of the patient; and determining, by the processing circuitry and based on the detected motion data, at least one of a gait or a posture of the patient, wherein the determined characteristics of the patient further comprise the at least one of the gait or the posture of the patient.

Example 31A: a computer readable storage medium comprising instructions that, when executed, cause processing circuitry within a vestibular implant system to perform a method of any of examples 16A-30A.

Example 32A: a device for vestibular stimulation, the device comprising: means for detecting movements of a head of a patient; means for detecting electrical signals in tissue of the patient; means for determining, based on the detected movements of the head, an angular shift frequency of the head; means for determining, based on the detected electrical signals, an evoked electrical signal in the tissue of the patient in response to the movements of the head; means for selecting, based on determined characteristics of the patient comprising the angular shift frequency and the evoked electrical stimulation signal, an operating mode for a vestibular implant of the vestibular implant system from a plurality of operating modes stored in memory of the vestibular implant system; and means for transmitting an electrical stimulation signal to one or more regions of the vestibular organ of an inner ear of the patient, the electrical stimulation signal having a stimulation pattern corresponding to the selected operating mode.

Example 33A: the device of example 31A, further comprising means for performing the method of any of examples 17A-30A.

Example 1B: A vestibular implant system comprising: a memory; an implantable sensor; a vestibular implant; and processing circuitry coupled to the memory, the processing circuitry configured to: detect, via the implantable sensor, an electrical signal of a patient that is generated in response to an electrical stimulation signal transmitted by the vestibular implant to one or more regions of vestibular organ of a patient; determine, based on the detected electrical signal, that the vestibular implant is transmitting the electrical stimulation signal at a first rate of change of an amplitude of the electrical stimulation signal; and based on a determination that the first rate of change satisfies a threshold condition, adjust one or more parameters of the electrical stimulation signal to cause the vestibular implant to transmit the electrical stimulation signal to the nerve at a second rate of change of the amplitude of the electrical stimulation signal.

Example 2B: the system of example 1B, wherein the processing circuitry is further configured to: determine, based on one or more of the detected electrical signal or the one or more parameters of the electrical stimulation signal, one or more signal saturation metrics; and based on the determination that at least one of one or more signal saturation metrics satisfies a corresponding signal saturation threshold condition, adjust the one or more parameters of the electrical stimulation signal to cause the vestibular implant to transmit the electrical stimulation signal to the nerve at the second rate of change of the amplitude.

Example 3B: the system of any of examples 1B and 2B, wherein the electrical signal comprises a current in a nerve.

Example 4B: the system of any of examples 1B-3B, wherein the one or more parameters of the electrical stimulation signal comprises one or more of a target amplitude or a frequency of the electrical stimulation signal.

Example 5B: the system of any of examples 1B-4B, wherein the processing circuitry is further configured to adjust the one or more parameters of the electrical stimulation signal based at least in part on a posture or a gait of the patient.

Example 6B: the system of any of examples 1B-5B, wherein the second rate of change of the amplitude of the electrical stimulation signal corresponds to a rate of change in one or more metrics corresponding to changes in movement of the patient.

Example 7B: the system of example 6B, wherein the one or more metrics comprises one or more of a myogenic potential in tissue of the patient or electrical activity in the nerve of the patient.

Example 8B: the system of example 7B, wherein the threshold condition corresponds to the one or more metrics corresponding to the changes in the movement of the patient.

Example 9B: the system of any of examples 2B-8B, wherein the one or more signal saturation metrics comprises a signal saturation status corresponding to whether the vestibular implant is transmitting the electrical stimulation signal.

Example 10B: the system of any of examples 2B-9B, wherein the one or more signal saturation metrics comprises a signal saturation degree, the signal saturation degree corresponding to an amplitude of the electrical stimulation signal relative to a maximum allowable amplitude of the electrical stimulation signal.

Example 11B: the system of any of examples 2B-10B, wherein the one or more signal saturation metrics comprises a signal saturation decay, the signal saturation decay corresponding to a rate of decay of the electrical stimulation signal in the tissue of the patient after the vestibular implant terminates the transmission of the electrical stimulation signal.

Example 12B: the system of example 11B, wherein the signal saturation threshold condition comprises a signal saturation decay profile of the patient.

Example 13B: the system of example 12B, wherein the processing circuitry is further configured to determine the signal saturation decay profile using a machine learning technique.

Example 14B: a method comprising: transmitting, via a vestibular implant of a vestibular implant system, an electrical stimulation signal to one or more regions of vestibular organ of a patient; detecting, via an implantable sensor of the vestibular implant system, an electrical signal of a patient in response to the transmitted electrical stimulation signal; determining, by processing circuitry of the vestibular implant system and based on the detected electrical signal, that the vestibular implant is transmitting the electrical stimulation signal to a nerve at a first rate of change of amplitude of the electrical stimulation signal; determining, by the processing circuitry, whether the first rate of change of the amplitude satisfies a threshold condition; and adjusting, based on the determination that the first rate of change of the amplitude satisfies the threshold condition, one or more parameters of the electrical stimulation signal to cause the vestibular implant to transmit the electrical stimulation signal to the nerve at a second rate of change of the amplitude.

Example 15B: the method of example 14B, further comprising: determining, by the processing circuitry and based on one or more of the detected electrical signal or the one or more parameters of the electrical stimulation signal, one or more signal saturation metrics; determining, by the processing circuitry, whether at least one of the one or more signal saturation metrics satisfies a corresponding signal saturation threshold condition; and adjusting, based on the determination that at least one of the one or more signal saturation metrics satisfies the corresponding signal saturation threshold condition, the one or more parameters of the electrical stimulation signal to cause the vestibular implant to transmit the electrical stimulation signal to the nerve at the second rate of change of the amplitude.

Example 16B: the method of any of examples 14B and 15B, wherein the one or more parameters of the electrical stimulation signal comprises one or more of a target amplitude or a frequency of the electrical stimulation signal.

Example 17B: the method of any of examples 14B-16B, further comprising: adjusting, by the processing circuitry, the one or more parameters of the electrical stimulation signal based at least in part on a posture or a gait of the patient.

Example 18B: the method of any of examples 14B-17B, wherein the second rate of change of the amplitude corresponds to a rate of change in one or more metrics corresponding to changes in movement of the patient.

Example 19B: the method of example 18B, wherein the one or more metrics comprises one or more of a myogenic potential in tissue of the patient or electrical activity in the nerve of the patient.

Example 20B: the method of example 19B, wherein the threshold condition corresponds to the one or more metrics corresponding to the changes in the movement of the patient.

Example 21B: the method of any of examples 15B-20B, wherein the one or more signal saturation metrics comprises a signal saturation status corresponding to whether the vestibular implant is transmitting the electrical stimulation signal.

Example 22B: the method of any of examples 15B-21B, wherein the one or more signal saturation metrics comprises a signal saturation degree, the signal saturation degree corresponding to an amplitude of the electrical stimulation signal relative to a maximum allowable amplitude of the electrical stimulation signal.

Example 23B: the method of any of examples 15B-22B, wherein the one or more signal saturation metrics comprises a signal saturation decay, the signal saturation decay corresponding to a rate of decay of the electrical stimulation signal in the tissue of the patient after the vestibular implant terminates the transmission of the electrical stimulation signal.

Example 24B: the method of example 23B, wherein the signal saturation threshold condition comprises a signal saturation decay profile of the patient.

Example 25B: the method of example 24B, further comprising: determining, by the processing circuitry, the signal saturation decay profile using a machine learning technique.

Example 26B: a vestibular implant system comprising: a vestibular implant; and a plurality of electrical leads, each of the plurality of electrical leads comprising: an elongated body extending from a proximal end to a distal end; an electrical conductor disposed on the proximal end of the elongated body and connected to the vestibular implant; an electrode disposed on the distal end of the elongated body; and an attachment device disposed on the distal end of the elongated body, wherein the attachment device is configured to affix the electrode to a semicircular canal of a patient at an implantation site on the semicircular canal.

Example 27B: the system of example 26B, wherein each of the plurality of electrical leads further comprises a marker disposed on the distal end of the elongated body, the marker configured to indicate a position of the electrode within body of the patient.

Example 28B: the system of example 27B, wherein the marker comprises a biocompatible material.

Example 29B: the system of any of examples 27B and 28B, wherein the marker comprises a radiopaque band.

Example 30B: the system of any of examples 27B-29B, wherein the marker comprises a coating disposed around the one or more of the electrode, the attachment device, or the distal end of the elongated body.

Example 31B: the system of any of examples 26B-30B, wherein the attachment device comprises a penetrating instrument configured to puncture tissue of the semicircular canal.

Example 32B: the system of example 31B, wherein the attachment device further comprises one or more protrusions extending from the penetrating instruments and configured to anchor the attachment device within the puncture.

Example 33B: the system of any of examples 26B-30B, wherein the attachment device comprises an adherence tool comprising a plurality of protrusions configured to be attached to tissue of the semicircular canal without puncturing the tissue.

Example 34B: the system of example 33B, wherein the plurality of protrusions is configured to attach the adherence tool to the issue when force is applied in a first direction and configured to resist attachment of the adherence tool to the tissue when force is applied in a second direction.

Example 35B: the system of example 34B, wherein the second direction is in an opposite direction as the first direction.

Example 36B: the system of any of examples 33B-35B, wherein one or more of the adherence tool or the plurality of protrusions comprises a metallic material.

Example 37B: the system of any of examples 33B-36B, wherein one or more of the adherence tool or the plurality of protrusions comprises a corrugated polymer.

Example 38B: the system of any of examples 26B-30B, wherein the attachment device comprises an adhesive, wherein the adhesive is configured to be disposed on an outer surface of the semicircular canal, and wherein the adhesive is configured to harden and affix the electrode to the outer surface of the semicircular canal.

Example 39B: the system of example 38B, wherein the adhesive comprises hydroxyapatite.

Example 40B: a method comprising: navigating a drill to temporal bone of a patient; forming a channel in the temporal bone to a recess surrounding a semicircular canal of the patient; advancing a catheter of a vestibular implant system through the channel and into the recess; disposing, via the catheter, an electrical lead within the recess, wherein the electrical lead is connected to a vestibular implant; and affixing the electrical lead to the base of the semicircular canal.

Example 41B: the method of example 40B, wherein the electrical lead is disposed within an inner lumen defined by the catheter.

Example 42B: the method of any of examples 40B and 41B, wherein the electrical lead comprises: an elongated body extending from a proximal end to a distal end; an electrical conductor disposed on the proximal end of the elongated body and connected to the vestibular implant; an electrode disposed on the distal end of the elongated body; and an attachment device disposed on the distal end of the elongated body.

Example 43B: the method of example 42B, wherein affixing the electrical lead to the base of the semicircular canals comprises affixing, via the attachment device, the distal end of the elongated body of the electrical lead to the base of the semicircular canal.

Example 44B: the method of any of examples 42B and 43B, wherein the electrical lead further comprises a marker disposed on the distal end of the elongated body, the marker configured to indicate a position of the distal end within body of the patient.

Example 45B: the method of any of examples 42B-44B, wherein the attachment device comprises a penetrating instrument configured to puncture tissue at the base of the semicircular canal.

Example 46B: the method of any of examples 42B-44B, wherein the attachment device comprises an adherence tool comprising a plurality of protrusions configured to be attached to tissue at the base of the semicircular canal without puncturing the tissue.

Example 47B: the method of any of examples 42B-44B, wherein the attachment device comprises an adhesive configured to be disposed on an outer surface of the base of the semicircular canal, and wherein the adhesive is configured to harden and affix the electrode to the outer surface of the base of the semicircular canal.

Example 48B: the method of example 47B, wherein the adhesive comprises hydroxyapatite.

Example 49B: the method of any of examples 40B-48B, wherein disposing, the electrical lead within the recess comprises navigating the electrical lead via a guide member disposed within the catheter.

Example 50B: the method of example 49B, wherein the guide member comprises a stylet.

Example 51B: the method of any of examples 40B-50B, wherein the recess surrounds all semicircular canals of the patient.

Various examples have been described. These and other examples are within the scope of the following claims.

What is claimed is:

1. A vestibular implant system comprising:
a memory;
an implantable sensor;
a vestibular implant; and
processing circuitry coupled to the memory, the processing circuitry configured to:
detect, via the implantable sensor, an electrical signal of a patient that is generated in response to an electrical stimulation signal transmitted by the vestibular implant to one or more regions of vestibular organ of a patient;
determine, based on the detected electrical signal, that the vestibular implant is transmitting the electrical stimulation signal at a first rate of change of an amplitude of the electrical stimulation signal; and
based on a determination that the first rate of change satisfies a threshold condition, adjust one or more parameters of the electrical stimulation signal to cause the vestibular implant to transmit the electrical stimulation signal to the nerve at a second rate of change of the amplitude of the electrical stimulation signal.

2. The system of claim 1, wherein the processing circuitry is further configured to:
determine, based on one or more of the detected electrical signal or the one or more parameters of the electrical stimulation signal, one or more signal saturation metrics; and
based on the determination that at least one of one or more signal saturation metrics satisfies a corresponding signal saturation threshold condition, adjust the one or more parameters of the electrical stimulation signal to cause the vestibular implant to transmit the electrical stimulation signal to the nerve at the second rate of change of the amplitude.

3. The system of claim 2, wherein the one or more signal saturation metrics comprises a signal saturation status corresponding to whether the vestibular implant is transmitting the electrical stimulation signal.

4. The system of claim 2, wherein the one or more signal saturation metrics comprises a signal saturation degree, the signal saturation degree corresponding to an amplitude of the electrical stimulation signal relative to a maximum allowable amplitude of the electrical stimulation signal.

5. The system of claim 2, wherein the one or more signal saturation metrics comprises a signal saturation decay, the signal saturation decay corresponding to a rate of decay of the electrical stimulation signal in the tissue of the patient after the vestibular implant terminates the transmission of the electrical stimulation signal.

6. The system of claim 5, wherein the signal saturation threshold condition comprises a signal saturation decay profile of the patient.

7. The system of claim 1, wherein the one or more parameters of the electrical stimulation signal comprises one or more of a target amplitude or a frequency of the electrical stimulation signal.

8. The system of claim 1, wherein the processing circuitry is further configured to adjust the one or more parameters of the electrical stimulation signal based at least in part on a posture or a gait of the patient.

9. The system of claim 1, wherein the second rate of change of the amplitude of the electrical stimulation signal corresponds to a rate of change in one or more metrics corresponding to changes in movement of the patient.

10. The system of claim 9, wherein the one or more metrics comprises one or more of a myogenic potential in tissue of the patient or electrical activity in the nerve of the patient.

11. A method comprising:
transmitting, via a vestibular implant of a vestibular implant system, an electrical stimulation signal to one or more regions of vestibular organ of a patient;
detecting, via an implantable sensor of the vestibular implant system, an electrical signal of a patient in response to the transmitted electrical stimulation signal;
determining, by processing circuitry of the vestibular implant system and based on the detected electrical signal, that the vestibular implant is transmitting the electrical stimulation signal to a nerve at a first rate of change of amplitude of the electrical stimulation signal;
determining, by the processing circuitry, whether the first rate of change of the amplitude satisfies a threshold condition; and
adjusting, based on the determination that the first rate of change of the amplitude satisfies the threshold condition, one or more parameters of the electrical stimulation signal to cause the vestibular implant to transmit the electrical stimulation signal to the nerve at a second rate of change of the amplitude.

12. The method of claim 11, further comprising:
determining, by the processing circuitry and based on one or more of the detected electrical signal or the one or more parameters of the electrical stimulation signal, one or more signal saturation metrics;
determining, by the processing circuitry, whether at least one of the one or more signal saturation metrics satisfies a corresponding signal saturation threshold condition; and
adjusting, based on the determination that at least one of the one or more signal saturation metrics satisfies the corresponding signal saturation threshold condition, the one or more parameters of the electrical stimulation signal to cause the vestibular implant to transmit the electrical stimulation signal to the nerve at the second rate of change of the amplitude.

13. The method of claim 12, wherein the one or more signal saturation metrics comprises a signal saturation status corresponding to whether the vestibular implant is transmitting the electrical stimulation signal.

14. The method of claim 12, wherein the one or more signal saturation metrics comprises a signal saturation degree, the signal saturation degree corresponding to an amplitude of the electrical stimulation signal relative to a maximum allowable amplitude of the electrical stimulation signal.

15. The method of claim 12, wherein the one or more signal saturation metrics comprises a signal saturation decay, the signal saturation decay corresponding to a rate of decay of the electrical stimulation signal in the tissue of the patient after the vestibular implant terminates the transmission of the electrical stimulation signal.

16. A vestibular implant system comprising:
a vestibular implant; and
a plurality of electrical leads, each of the plurality of electrical leads comprising:
an elongated body extending from a proximal end to a distal end;
an electrical conductor disposed on the proximal end of the elongated body and connected to the vestibular implant;
an electrode disposed on the distal end of the elongated body; and
an attachment device disposed on the distal end of the elongated body, wherein the attachment device is configured to affix the electrode to a semicircular canal of a patient at an implantation site on the semicircular canal.

17. The system of claim 16, wherein the attachment device comprises:
a penetrating instrument configured to puncture tissue of the semicircular canal; and
one or more protrusions extending from the penetrating instruments and configured to anchor the attachment device within the puncture.

18. The system of claim 16, wherein the attachment device comprises an adherence tool comprising a plurality of protrusions configured to be attached to tissue of the semicircular canal without puncturing the tissue.

19. The system of claim 18, wherein the plurality of protrusions is configured to attach the adherence tool to the issue when force is applied in a first direction and configured to resist attachment of the adherence tool to the tissue when force is applied in a second direction.

20. The system of claim 16, wherein the attachment device comprises an adhesive, wherein the adhesive is configured to be disposed on an outer surface of the semicircular canal, and wherein the adhesive is configured to harden and affix the electrode to the outer surface of the semicircular canal.
